(19) United States
(12) Patent Application Publication
(10) Pub. No.: US 2008/0302732 A1
(43) Pub. Date: Dec. 11, 2008

(54) INTEGRATED FLUIDICS DEVICES WITH MAGNETIC SORTING

(76) Inventors: Hyongsok Soh, Santa Barbara, CA (US); Brian Scott Ferguson, Goleta, CA (US); Yanting Zhang, Goleta, CA (US); Nancy Stagliano, Santa Barbara, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 12/126,670

(22) Filed: May 23, 2008

Related U.S. Application Data
(60) Provisional application No. 60/931,797, filed on May 24, 2007.

(51) Int. Cl. B03C 1/30; G01N 33/48

(52) U.S. Cl. 210/695; 422/73

(57) ABSTRACT

A fluidic device employs one or more sorting stations for separating target species from other species in a sample. At least one of the sorting stations employs a magnetic field gradient to accomplish separation. In addition, the sorting station is integrated on a single substrate with one or more other modules for processing the sample. For example, the fluidic device may include both a sorting station and a separate trapping station that holds some or all components of the sample for additional processing. The trapping station may be located at a position upstream or downstream from the sorting module.

### CROSS REFERENCE TO RELATED APPLICATIONS

This application claims priority to U.S. Provisional Patent Application No. 60/931,797 filed May 24, 2007, which is incorporated herein by reference in its entirety and for all purposes.

### BACKGROUND

Sorting cells based on their surface markers is an important capability in biology and medicine. Magnetic cell sorting techniques allow rapid selection of a large number of target cells. The applications of such techniques span a broad spectrum, ranging from protein purification to cell based therapies. In some approaches, target cells are labeled through a superparamagnetic particle that is conjugated to a molecular recognition element (e.g. a monoclonal antibody) which recognizes a particular cell surface marker.

Current systems are capable of high-purity selection of the labeled cells. However, they often suffer in terms of throughput and recovery. Certain applications such as recovery of rare cells demand higher recovery than many available systems provide. In order to achieve higher throughput and higher recovery of the rare cells (or other target components), improvements on existing systems are needed.

### SUMMARY

Embodiments of the invention provide a fluidic device employing one or more sorting stations for separating target species from other species in a sample. At least one of the sorting stations employs a magnetic field gradient to accomplish separation. In addition, the sorting station is integrated on a single substrate with one or more other modules for processing the sample. For example, the fluidic device may include both a sorting station and a separate trapping station that holds some or all components of the sample for additional processing. The trapping station may be located at a position upstream or downstream from the sorting module.

In certain embodiments, a fluidic sorting device may be characterized by the following features: (a) a substrate comprising a plurality of fluidic modules; (b) a sorting station comprising a magnetic field concentrator for exerting magnetic force on a sample in a fluid medium to divert magnetic particles in the sample and thereby sort magnetic and non-magnetic entities in the fluid medium; (c) a trapping module fluidically coupled to the sorting station and located downstream therefrom; and (d) an additional module fluidically coupled to the trapping module and located downstream therefrom. The sorting station, the trapping module, and the additional module are each provided on the single substrate. Further, in certain embodiments, the trapping module includes (i) a valve for controlling flow of the fluid medium between the sorting station and the trapping module, (ii) an inlet for delivering a reagent, and (iii) a structure for capturing and immobilizing, at least temporarily, magnetic particles or a particular species in the sample. The trapping module may be configured to perform one or more of the following functions: releasing the magnetic particles from sample species specifically bound to the magnetic particles, and washing trapped species in the sample. As used herein, the terms “specifically bound,” “specific binding” and the like refer to interactions that occur between binding partners such that the binding partners bind preferentially to one another relative to other entities that may be present in the environment (e.g., in a fluid sample). In certain embodiments, the additional module includes a valve for controlling flow of the fluid medium between the trapping module and the additional module and is configured to perform one or more of the following functions: species detection and nucleic acid amplification. For many applications, fluidic device is also outfitted with a controller coupled to the substrate for controlling and synchronizing the operations of the sorting station, the trapping module, and the additional module.

The fluidic sorting station may have at least one inlet channel for receiving the fluid medium with the magnetic particles, and at least one outlet channel for delivering the fluid medium enriched in or depleted of said magnetic particles. As used in this context, the term “enriched” means that the concentration of magnetic particles is higher in the fluid medium delivered from the at least one outlet channel than in the fluid medium received at the at least one inlet channel. In contrast, the term “depleted” indicates that the concentration of magnetic particles is lower in the fluid medium delivered from the at least one outlet channel than in the fluid medium received at the at least one inlet channel. The magnetic field concentrator in the sorting stations may have one or more elements that are made from a material having a permeability that is significantly different from that of the fluid medium in the device. As used in this context, the term “significantly different” means that the permeability of the material making up the one or more elements in the magnetic field concentrator is such that the elements, when exposed to a magnetic field, produce a magnetic field gradient sufficient to divert the magnetic particles in the fluid medium. For example, the magnetic field concentrator may have a pattern of ferromagnetic material arranged to interact with an external magnetic field and thereby produce a strong magnetic field gradient in the sorting station. Generally, this magnetic field gradient is greater than about 1 Tesla/meter and less than about 1 million Tesla/meter. For example, the magnetic field gradient can be greater than about 10 Tesla/meter and less than about 100,000 Tesla/meter, greater than about 100 Tesla/meter and less than about 10,000 Tesla/meter, or about 1000 Tesla/meter. In one embodiment, the average magnetic field gradient produced is about 200 Tesla/meter to about 10,000 Tesla/meter. In certain embodiments, the sorting station has one inlet configured to deliver buffer to the sorting station and another inlet configured to deliver sample to sorting station.

The trapping module in the fluidic device may employ any one or more of many different types of mechanisms to trap species from the sample. In some embodiments, the trapping module includes ferromagnetic structures for concentrating a magnetic field in the trapping module. In some designs, the ferromagnetic structures are located preferentially toward an outlet to the trapping module.

While trapped in the trapping module, the species are processed in some manner. For example, the trapping module may be designed or configured so that the magnetic particles, while they are trapped in the module, are released from sample species to which they are specifically bound. In another example, the trapped species are washed or further concentrated. Further, the species may contain a nucleic acid which is amplified while the species reside in the trapping module. In a specific embodiment, the trapping module is configured to perform lysis of trapped species from the sample and subsequently perform PCR on nucleic acids released during the lysis.

One example of the reagent for use in the trapping module is a buffer. Other examples include PCR reagents, reagents for releasing magnetic particles from the sample components, reagents for lysing trapped cells or viruses, etc. Also, the trapping station may be configured to label the trapped species from the sample. In such designs, the reagent may be a label such as, e.g., a fluorescent label that specifically binds to an antigen expressed on the surface of a target species.

The additional module may be designed or configured to perform any one or more of several appropriate processes. For example, in certain embodiments, the additional module in the fluidic device may be a detector. As another example, the additional module is configured to perform a nucleic acid amplification technique.

Also disclosed herein, is a microfluidic sorting device comprising: (a) a magnetic field gradient generator for exerting a magnetic force on a sample to divert magnetic particles in the sample and thereby separate them from other components; and (b) a pre-processing and/or a post-processing trapping station integrated on the microfluidic sorting device with the magnetic field gradient generator.

The pre-processing station can include a labeling station for labeling target species in the sample with magnetic particles capable of specifically binding to the target species. The post-processing station can include a detection station for detecting the target species.

In one embodiment, at least one of the pre-processing station or the post-processing station comprises one or more of the following: (a) an enrichment module, (b) a reaction module, (c) a detection module, and (d) a lysis module for lysing cells or disrupting viral protein coats. In another embodiment, at least one of the pre-processing station or the post-processing station is designed or configured to perform genomic analysis, amplification of a nucleic acid, gene expression analysis, enzymatic activity assays, receptor binding assays, or ELISA assays.

Another aspect of the invention pertains to methods of processing a sample. Such methods may be characterized by the following operations: (a) providing the sample to a fluidic sorting device on a substrate, wherein the sample includes magnetic particles capable of specifically binding one or more species suspected of being contained in the sample; (b) magnetizing a magnetic field concentrator to divert and thereby concentrate the magnetic particles in the sample; (c) delivering the magnetic particles and bound species, if any, from the sample to a trapping module; (d) washing magnetic particles and/or releasing bound species, if any, from the sample while in the trapping module; and (e) delivering the bound species, if any, to an additional module where the bound species, if any, are detected and/or reacted with a reagent.

In certain embodiments the method includes, prior to (a), labeling the one or more species, if any, with the magnetic particles. In some cases, the method also involves performing one or more of the following operations in the trapping module: nucleic acid amplification, a restriction enzyme reaction, nucleic acid sequencing, target labeling, chromatin immunoprecipitation, crosslinking, and cell culture.

The method may also involve performing one or more other operations in the additional module. For example, nucleic acids associated with the bound species, if any, may be amplified. In another example, the sample species, if any, may be lysed in the additional module.

These and other features and advantages of the invention will be presented in further detail below with reference to the associate drawings.

### BRIEF DESCRIPTION OF THE DRAWINGS

FIG. 1 is a schematic illustration of a trapping station that may be provided downstream from a magnetophoretic sorting station.

FIG. 2 is an illustration showing certain design options for magnetic trapping chambers in accordance with some embodiments of this invention.

FIG. 3A is a schematic depiction of one type of magnetic deflection chamber in a microfluidic device.

FIG. 3B is a schematic depiction of another type of magnetic deflection chamber in a microfluidic device.

FIG. 4 is a top view of the channels and magnetic field gradient generating structures in one example of a magnetophoretic microfluidics device.

FIG. 5 is a schematic depiction of a weir type trapping module.

FIG. 6 is a schematic depiction of another weir type trapping module.

FIG. 7 is a schematic depiction of one type of optical trapping module.

FIG. 8A is an illustration showing the fabrication and assembly of a dielectrophoretic PCR module in accordance with an embodiment.

FIG. 8B is an illustration showing a chip containing the DEPCR module of FIG. 8A in an apparatus during operation.

FIG. 8C is a microscopic view of electrodes trapping bacteria in the dielectrophoretic module of FIG. 8A.

FIG. 8D is a gel after running the post-PCR sample from the DEPCR module of FIG. 8A and beside certain controls.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, to any specifically excluded limit in the stated range. Where the stated range includes one or both of the limits, ranges excluding either or both of those included limits are also included in the invention.

It must be noted that as used herein and in the appended claims, the singular forms “a,” “an,” “and,” and “the” include plural referents unless the context clearly dictates otherwise. Thus, for example, reference to “a magnetic particle” includes a plurality of such magnetic particles and reference to “the trapping module” includes reference to one or more trapping modules and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DESCRIPTION OF A PREFERRED EMBODIMENT

Introduction

Magnetic sorting apparatuses may be integrated with various other operational modules in a fluidics system. Generally a magnetic sorting apparatus employs a magnetic field gradient to exert a force on magnetic particles flowing in a fluid medium. Under the influence of a magnetic force, the magnetic particles are diverted from their normal trajectory, which may be dictated by hydrodynamic forces acting on the particles. Other forces such as gravitational forces may also influence the path of the particles. The magnetic field gradient may be produced by many different types of apparatus. In a typical embodiment, an external magnet (i.e., a magnet located externally to a fluidics chip applies a magnetic field in a magnetic sorting station formed on the chip. A magnetic field concentrator is typically employed to increase the local field gradient in an area where the magnetic particles are located or flow into. One example of a magnetic field concentrator includes one or more gradient shaping elements disposed on or proximate to the sorting station. Such elements are made from a material having a permeability that is significantly different from that of the fluid medium in the device (e.g., an aqueous buffer). In certain cases, the elements are made from a ferromagnetic material. In a specific embodiment, the patterns are defined by nickel features on a glass or polymer substrate, which is the substrate on which the fluidics chip is formed.

In certain embodiments, the sorting stations are magnetophoretic sorting modules employing magnetic field gradient generators (MFGs). Such sorting modules may employ buffer switching as described in U.S. patent application Ser. No. 11/583,989 filed Oct. 18, 2006, which disclosure is incorporated herein by reference. However, in the following description, it is assumed that references to magnetic or magnetophoretic sorters or sorting modules encompass various types of sorting modules, not necessarily those relying on magnetophoretic separation. Often, the described sorting modules may employ MFGs and/or buffer switching.

The integrated systems described herein are assumed to include two or more processing stations or operational modules, e.g., 3 or more, 4 or more, 5 or more, 6 or more, etc., each configured to perform a separate unit operation. In many embodiments, at least one of these operational modules is a magnetophoretic sorting module. In many embodiments, the two or more operational modules are fabricated on a single substrate (e.g., a monolithic piece of glass or silicon). In other words, the modules are integrated on a single fluidics chip.

Examples of operational modules that may be integrated with magnetophoretic sorters in fluidics devices include (a) enrichment modules such as fluorescence activated cell sorters, washing modules, and various types of traps, (b) reaction modules such as sample amplification (e.g., PCR) modules, restriction enzyme reaction modules, nucleic acid sequencing modules, target labeling modules, chromatin immunoprecipitation modules, crosslinking modules, and even cell culture modules, (c) detection modules such as microarrays of nucleic acids, antibodies or other highly specific binding agents, and fluorescent molecular recognition modules, and (d) lysis modules for lysing cells, disrupting viral protein coats, or otherwise releasing components of small living systems. Each of these modules may be provided before or after the magnetic sorter. There may be multiple identical or different types of operational modules integrated with a magnetic sorter in a single fluidics system. Further, one or more magnetic sorters may be arranged in parallel or series with respect to various other operational modules. Various of the operational modules may be designed or configured as traps in which target species in a sample are held stationary or generally constrained in a particular volume.

As should be apparent from the above examples of modules, operations that may be performed on target and/or non-target species in modules of integrated fluidics devices include sorting, binding, washing, trapping, amplifying, removing unwanted species, precipitating, cleaving, diluting, ligating, sequencing, synthesis, labeling (e.g., staining cells), cross-linking, culturing, detecting, imaging, quantifying, lysing, etc.

Specific examples of biochemical operations that may be performed before or after magnetic sorting in integrated fluidic devices include synthesis and/or screening of plasmids, aptamers, proteins, and peptides; evaluating enzyme activity; and derivatizing proteins and carbohydrates. A broad spectrum of biochemical and electrophysiological assays may also be performed, including: (1) genomic analysis (sequencing, hybridization), PCR and/or other detection and amplification schemes for DNA, and RNA oligomers; (2) gene expression; (3) enzymatic activity assays; (4) receptor binding assays; and (5) ELISA assays. The foregoing assays may be performed in a variety of formats, such as: homogeneous, bead-based, and surface bound formats. Furthermore, devices as described herein may be utilized to perform continuous production of biomolecules using specified enzymes or catalysts, and production and delivery of biomolecules or molecules active in biological systems such as therapeutic agents. Microfluidic devices as described herein may also be used to perform combinatorial syntheses of peptides, proteins, and DNA and RNA oligomers as currently performed in macrofluidic volumes.

It should be understood that embodiments of the invention are not limited to analysis of biological or even organic materials, but extend to non-biological and inorganic materials. Thus, the apparatus and methods described above can also be used to screen or otherwise analyze non-biological substances in liquids. The analyte and target species may comprise small or large chemical entities of natural or synthetic origin such as chemical compounds, supermolecular assemblies, fragments, glasses, ceramics, etc. In certain embodiments, they are monomers, oligomers, and/or polymers having any degree of branching. They may be expressed on a cell or virus or they may be independent entities. They may also be complete cells or viruses themselves.

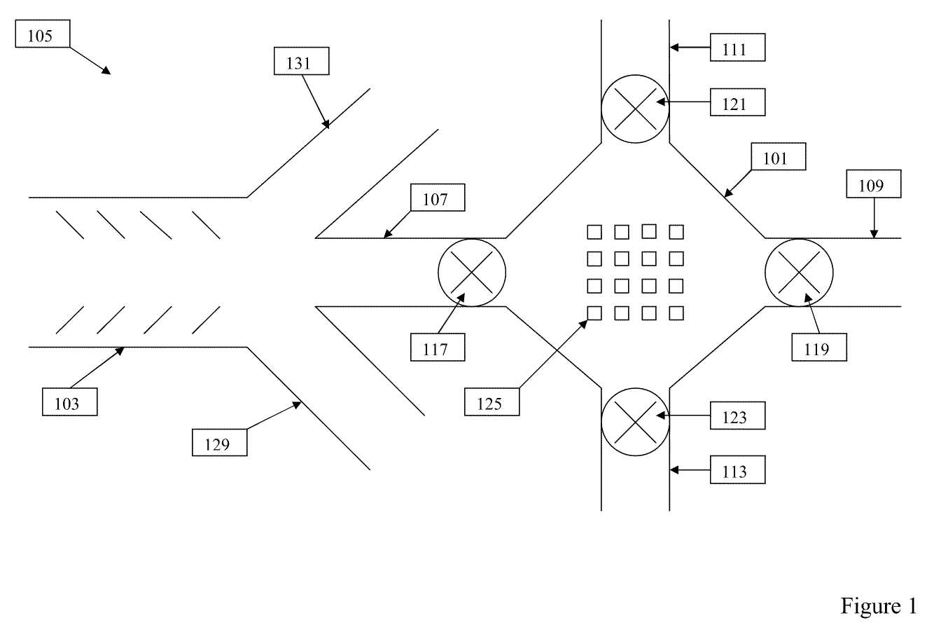

As indicated, one or more traps may be employed as operational modules integrated with sorting modules (e.g., magnetophoretic sorters) in on-chip fluidics systems. Examples of types of traps include optical traps, magnetic traps, electrostatic traps, mechanical traps (e.g., weirs), acoustic traps, etc. Some of these will be described in more detail below. FIG. 1 shows a general non-limiting structure of a trap 101 disposed downstream from magnetic sorter module 103 and upstream from another processing module (not shown) in an integrated fluidics device 105. As shown, trap 101 includes an inlet line 107 for receiving a concentrated sample stream from sorter 103 and an outlet line 109. Trap 101 also includes auxiliary lines 111 and 113 for providing one or more other reagents. Each of lines 107, 109, 111, and 113 includes its own valve 117, 119, 121, and 123, respectively. Within trap 101 are various trapping elements 125. These may be ferromagnetic elements that shape or deliver a magnetic field, electrodes, regions of immobilized target binding agents, oscillators, piezoelectric elements, etc. In FIG. 1, the sorting module is shown as including two waste stream outlets 129 and 131.

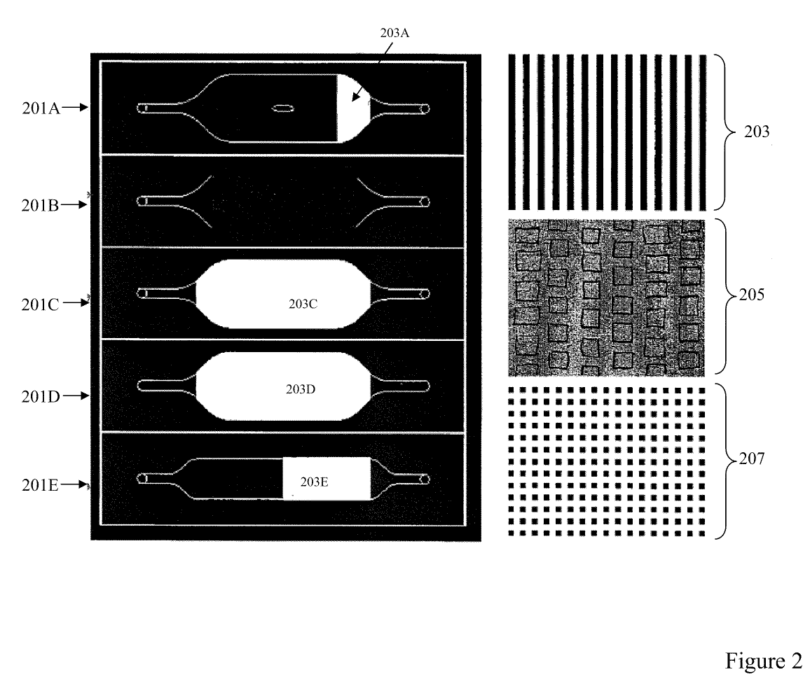

FIG. 2 depicts magnetic trap structures that may be employed with certain embodiments of this invention. In these embodiments, three different ferromagnetic element patterns are shown on the right side of the figure. These are employed to shape a magnetic field gradient originating from an external source of a magnetic field (not shown). In a first embodiment, a pattern 203 employs parallel stripes to shape a magnetic field gradient. In a second embodiment, a pattern 205 employs relatively large square regions to shape the magnetic field gradient. Finally, in a third embodiment, a pattern 207 employs relatively small squares to shape the gradient. The smaller features have dimensions (length and width on the surface of the substrate) on the order of about 10 micrometers. These features or elements in these patterns may be made from various materials. Generally, the elements are made from a material having a permeability that is significantly different from that of the fluid medium in the device (e.g., the buffer). In certain cases, the elements will be made from a ferromagnetic material. In a specific embodiment, the patterns are defined by nickel features on a glass or polymer substrate. In alternative embodiments, the patterns shown in these embodiments present other types of capture structures such as electrodes, specific binding moieties (e.g., regions of nucleotide probes or antibodies), physical protrusions or indentations, etc.

On the left side of FIG. 2 are five different trapping chamber designs 201A-201E, illustrating the relative positions of the capture pattern in the chamber. Fluid is assumed to flow from left to right in these figures. The regions 203A, and 203C-203E represent the positions of the patterns within the chambers. In any of the three trap chamber layouts, any one or more of three patterns shown on the right may be used. Of course, other patterns may be employed as well. Note that in the top and bottom trap chamber designs depicted in FIG. 2, the capture structures are located at the downstream side of the chamber. Such designs may take advantage of self-magnetization of captured magnetic particles. Initially, particles flowing into the chambers are captured by structures in the pattern. The captured particles are themselves magnetized and act as capture structures for subsequent magnetic particles flowing through the trap. Thus, a relative small magnetic capture region in a chamber may be sufficient to allow full utilization of the chamber to hold magnetic particles. Even after the pattern itself becomes saturated with captured particles, it can continue to capture additional particles using magnetized particles that were previously captured.

Typical sizes of trapping chambers are between about 1 and 100 millimeters in length (in the direction of flow), between about 1 and 100 millimeters in width and between about 1 micrometer and 10 millimeters depth (although typically about 1 millimeter or less). In more specific embodiments, the trapping chambers have a length of between about 5 and 70 millimeters. Independently, the chambers have widths of between about 3 and 50 millimeters. In certain embodiments, the chambers have an aspect ratio (length to width) that is greater than 1, e.g., about 2 to 8. In the depicted embodiments of FIG. 2, the aspect ratios are 3 and 5.

The applied magnetic field should be sufficiently great to capture or trap magnetic particles flowing in a fluid medium. Those of skill in the art will recognize that the applied magnetic force must be significantly greater than the force exerted on the particles by the flowing fluid.

In certain embodiments, the integrated fluidics systems are microfluidic systems. Microfluidic systems may be characterized by devices that have at least one “micro” channel. Such channels may have at least one cross-sectional dimension on the order of a millimeter or smaller (e.g., less than or equal to about 1 millimeter). For certain applications, this dimension may be adjusted; in some embodiments the at least one cross-sectional dimension is about 500 micrometers or less. In some embodiments, again as applications permit, a cross-sectional dimension of a given module or station (such as a sorting station or a trapping module) is about 100 micrometers or less (or even about 10 micrometers or less—sometimes even about 1 micrometer or less). A cross-sectional dimension is one that is generally perpendicular to the direction of centerline flow, although it should be understood that when encountering flow through elbows or other features that tend to change flow direction, the cross-sectional dimension under consideration need not be strictly perpendicular to flow. It should also be understood that in some embodiments, a micro-channel may have two or more cross-sectional dimensions such as the height and width of a rectangular cross-section or the major and minor axes of an elliptical cross-section. Either of these dimensions may be compared against sizes presented here. Note that micro-channels employed in this invention may have two dimensions that are significantly disproportionate, e.g., a rectangular cross-section having a height of about 100-200 micrometers and a width on the order of a centimeter or more. Of course, certain devices may employ channels in which the two or more axes are very similar or even identical in size (e.g., channels having a square or circular cross-section).

Often a controller will be employed to coordinate the operations of the various systems or sub-systems employed in the overall microfluidic system. Such controller will be designed or configured to direct the sample through a microfluidic flow passage. It may also control other features and actions of the system such as the strength and orientation of a magnetic field applied to fluid flowing through the microfluidic device or certain stations therein (e.g., the sorting and/or trapping stations), control of fluid flow conditions within the microfluidic device by actuating valves and other flow control mechanisms, mixing of magnetic particles and sample components in an attachment system, generating the sample (e.g., a library in a library generation system), and directing fluids from one system or device to another. The controller may include one or more processors and operate under the control of software and/or hardware instructions.

In certain embodiments, the interface between a magnetic sorter and another integrated module is lossless or nearly lossless. The recovery of target in a sample refers to the percentage of the target that is recovered. Typically, there will be losses but one advantage of the integrated fluidic devices disclosed herein is their ability to provide high recovery rates for rare species. In some examples, a lossless recovery may occur.

In some applications, loss of target species in a sample can lead to lack of commercial acceptance. Losses may be particularly problematic when the target is a rare cell species such as certain pathogens, tumor cells, stem cells, etc. As indicated, near 100% recovery of target species is sometimes desirable, even in cases where the initial concentration target in sample is low (e.g., no greater than about 1 percent) or extremely low (e.g., no greater than about 10⁻⁵ or even 10⁻⁷).

Many types of microvalves are known in the art and may be employed with this invention. Many of these are described in the review article K. W Oh and C. H. Ahn, A review of microvalves, J. Micromech. Microeng. 16 (2006) R13-R39, which is incorporated herein by reference for all purposes. Both active microvalves, using mechanical and non-mechanical moving parts, as well as external systems, and passive microvalves, using mechanical and non-mechanical moving parts may be used with this invention.

Active microvalves employ external energy or power sources (e.g., voltage or current) to drive a membrane or other structure into a fluid channel. Examples of mechanical actuation mechanisms for active microvalves include magnetic, electric, piezoelectric, and thermal. Magnetic actuators include those provided by external magnetic fields as well as integrated magnetic inductors. Electric actuators include those provided by electrostatic as well as electrokinetic mechanisms. Examples of thermal actuators include those employing bimetallic structures, thermopneumatic devices, and shape memory alloys. Mechanical active microvalves may be fabricated using the MEMS-based bulk or surface micromachining technologies, where mechanically movable membranes are coupled to magnetic, electric, piezoelectric or thermal actuation methods.

Nonmechanical active microvalves can be operated by the use of, for example, “smart materials.” These non-mechanical active microvalves may hold movable membranes which are actuated using functionalized smart materials such as phase change or rheological materials. Nonmechanical active valves may be driven by, for example, electrochemical reactions, phase changes (using, e.g., hydrogel, sol-gel, and paraffin based materials), and rheological materials (e.g., devices employing electro-rheological devices or ferrofluids).

Other categories of microvalve actuators may be used with this invention. For example, active microvalves may employ external systems. Such microvalves are actuated by the aid of external systems such as built-in modular or pneumatic means. As mentioned, examples of passive microvalves include both mechanical and nonmechanical devices. Examples of passive microvalves include check valves. Examples of nonmechanical valves include those employing diffusers and others employing capillary devices. Additionally, based on their base mode, microvalves may be divided into normally open, normally closed and bistable microvalves.

Often the most significant losses in microfluidic devices are in the delivery of the sample to the microfluidic devices as well as removal therefrom. Particular problems occur when the sample is transferred from one device to another. In such cases, the sample may pass through device interfaces that present changes in channel diameter, flow field, etc., each introducing its own losses. Further, in some cases the sample is provided via a pump or syringe when delivered to the device or through a pipette when removed from a device. Each of these operations may introduce significant losses. In integrated fluidics devices such as those described herein, losses are minimized by delivering sample directly between various operational modules on a single device. The “on-chip” interfaces between integrated modules may be engineered to reduce losses. Notably, in such designs, the sample is not passed through a pump, syringe, or tubing during transfer between the operational modules such as MFG sorting stations, labeling stations, detection stations, etc. It should also be understood that integration in the manner described herein allows analysis of very small samples, such as small numbers of beads or target species—e.g., on the order of 1000 or less. This is allowed by the fine control and sensitivity of the fluidics modules and high recovery (low loss) of target species.

Examples of Integrated Systems Employing Magnetic Sorters and Traps

Referring to the fluidics device 105 of FIG. 1, an example of a concentrator module will be described. In this embodiment, magnetic particles are sorted by MFG 103 and provided in a concentrated outlet stream to trap 101 via inlet channel 107. During this phase of the operation, valves 117 and 119 are open and valves 121 and 123 are closed. While a magnetic field or other capturing stimulus is provided via the trap features 125, the particles flowing into trap 101 are captured. After a sufficient number of particles are captured (which might be indicated by simply running a sample stream through device 105 for a defined period of time), valves 117 and 119 are closed. Thereafter, in one embodiment, valves 121 and 123 are opened, and a buffer is passed from line 111, through trap 101, and out line 113. This serves to wash the captured particles. After washing for a sufficient length of time, the washed particles may be recovered by eluting (by e.g., removing an external magnetic or electrical field while the buffer continues to flow), by pipetting from trap 101, by removing a lid or cover on the trap or the entire device, etc. Regarding the last option, note that in some embodiments the devices are disposable and can be designed so that the top portion or a cover is easily removed, e.g., by peeling.

In some embodiments, the trap includes a binding agent for capturing magnetic particles and/or the target component itself (or the non-target species if the magnetic particles happen to be coated with an agent for specifically binding non-target species). In some embodiments, the binding agent is coupled to the substrate (base or wall of the microfluidic device) via a linker. Suitable linkers can include cleavable linkers that can be attached to a solid support to permit release of a captured target species through cleavage of the cleavable linker. These cleavable linkers can be used to attach a variety of molecules to solid supports and arrays. For example, the cleavable linkers can be used to attach molecules such as glycans, nucleic acids or proteins to solid supports or arrays. The cleavage mechanism can be chemical, nuclease, protease, photolytic, etc. as known to those of skill in the art. Linker moieties susceptible to cleavage include disulfides, esters, nucleotides having a specified sequence, peptides having a specified sequence, etc. as known to those of skill in the art. In application, a component of a sample is captured by binding to a binding agent affixed to a trap or flow path in a microfluidic device. After appropriate treatment or analysis, the trap or flow path is exposed to a cleaving agent, which cleaves the linker holding the binding agent (and sample component) to the solid support. Thereafter, the previously bound sample component can be removed by flowing through an exit channel, by pipetting, etc.

In another embodiment, the particles that have been captured and washed in the trap as described above are exposed to one or more markers (e.g., labeled antibodies) for target cells or other target species in the sample. Certain tumor cells to be detected, for example, express two or more specific surface antigens. This combination of antigens occurs only in very unique tumors. To detect the presence of such cells bound to magnetic particles, valves 117 and 123 may be closed and valve 121 opened after capture in trap 101 is complete. Then a first label is flowed into trap 101 via line 111 and out via line 109. Some of the label may bind to immobilized cells in trap 101. Thereafter, valve 121 is closed and valve 123 is opened and a second label enters trap 101 via line 113. After label flows through the trap for a sufficient length of time, the captured particles/cells may be washed as described above. Thereafter, the particles/cells can be removed from trap 101 for further analysis or they may be analyzed in situ. For example, the contents of trap 101 may be scanned with probe beams at excitation for the first and second labels if such labels or fluorophores for example. Emitted light is then detected at frequencies characteristic of the first and second labels. In certain embodiments, individual cells or particles are imaged to characterize the contents of trap 101 and thereby determine the presence (or quantity) of the target tumor cells. Of course various target components other than tumor cells may be detected. Examples include pathogens such as certain bacteria or viruses.

In a related embodiment, the magnetic particles have a functionalized surface that specifically binds to one of the antigens on the tumor cell or other target species in a sample. Those sample components that do not possess the antigen do not bind with the magnetic particles and are therefore not selected in the sorting station. After the magnetic particles become trapped in the trapping station, they are exposed to a label for the second antigen and then washed. If the label for the second antigen is detected on species bound to the magnetic particles, one can assume that species having both antigens are present in the sample.

In yet another embodiment, the sample is treated in the following sequence. First it is contacted with magnetic particles as described in the previous embodiment, which have a functionalized surface that specifically binds to one of the antigens. Thereafter the magnetic particles, with bound species attached, are selected. Then the bound species may be optionally cleaved from the magnetic particles. Regardless of whether or not they are cleaved from the magnetic particles, they are next exposed to second magnetic particles which are functionalized with a surface material that binds specifically to the second antigen. These second magnetic particles are then selected. Any species attached to these second particles will harbor both antigens.

In another embodiment, nucleic acid from a sample enters trap 101 via line 107 and is captured by an appropriate mechanism (examples set forth below). Subsequently, valve 117 is closed and PCR reagents (nucleotides, polymerase, and primers in appropriate buffers) enter trap 101 via lines 111 and 113. Thereafter all valves (117, 119, 121, and 123) are closed and an appropriate PCR thermal cycling program is performed on trap 101. The thermal cycling continues until an appropriate level of amplification is achieved. Subsequently in situ detection of amplified target nucleic acid can be performed for, e.g., genotyping. Alternatively, the detection can be accomplished downstream of trap 101 in, e.g., a separate chamber which might contain a nucleic acid microarray or an electrophoresis medium. In another embodiment, real time PCR can be conducted in trap 101 by introducing, e.g., an appropriately labeled intercalation probe or donor-quencher probe for the target sequence. The probe could be introduced with the other PCR reagents (primers, polymerase, and nucleotides for example) via line 111 or 113. In situ real time PCR is appropriate for analyses in which expression levels are being analyzed. In either real time PCR or end point PCR, detection of amplified sequences can, in some embodiments, be performed in trap 101 by using appropriate detection apparatus such as a fluorescent microscope focused on regions of the trap.

For amplification reactions, various mechanisms may be employed to confine the nucleic acid sample to reaction chamber 101. For example, device 105 may be configured to process whole cells affixed to magnetic particles. In such cases, after the sample is sorted in chamber 103 and concentrated magnetic particles are delivered to trap 101, they are captured by elements 125, which may exert a magnetic field sufficient to capture the flowing particles. Thereafter, the flow through line 107 is shut off and a lysing agent (e.g., a salt or detergent) is delivered to chamber 101 via, e.g., line 111 or 113. The lysing agent may be delivered in a plug of solution and allowed to diffuse throughout chamber 101, where it lyses the immobilized cells in due course. This allows the cellular genetic material to be extracted for subsequent amplification. In certain embodiments, the lysing agent may be delivered together with PCR reagents so that after a sufficient period of time has elapsed to allow the lying agent to lyse the cells and remove the nucleic acid, a thermal cycling program can be initiated and the target nucleic acid detected.

In other embodiments, sample nucleic acid is provided in a raw sample and coupled to magnetic particles containing appropriate hybridization sequences. The magnetic particles are then sorted and immobilized in trap 101. After PCR reagents are delivered to chamber 101 and all valves are closed, PCR can proceed via thermal cycling. During the initial temperature excursion, the captured sample nucleic acid is released from the magnetic particles.

In certain embodiments, magnetic particles containing complementary nucleic acids affixed thereto are provided as a reagent to the trapping module after lysis. These particles are allowed to interact with the fluidic medium including the cellular materials released during lysis. Then the magnetic particles are immobilized in the trapping module by applying a strong external magnetic field to the module. Outlet valves are then opened and buffer flows through the trapping module to wash away at least some of the unbound cellular debris. After washing is completed, the outlet valve is then closed the nucleic acids from lysed cells or virus are then released from the captured magnetic particles and PCR or other amplification technique is performed without interference from unwanted cellular debris.

The nucleic acid amplification technique described herein is a polymerase chain reaction (PCR). However, in certain embodiments, non-PCR amplification techniques may be employed such as various isothermal nucleic acid amplification techniques; e.g., real-time strand displacement amplification (SDA), rolling-circle amplification (RCA) and multiple-displacement amplification (MDA). Each of these can be performed in a trap such as chamber 101 shown in FIG. 1.

While the examples set forth with respect to FIG. 1 show trapping, reactions, and/or detection/imaging being conducted in a chamber located downstream from the magnetic sorting module, this need not be the case. In many embodiments, a trapping chamber is disposed upstream from the sorting module. Such trapping modules may be employed for purposes of labeling magnetic particles with sample components, labeling or staining sample components, lysing sample components, etc. As with downstream trapping modules, capture may be accomplished using magnetic fields. Other capture techniques include optical, electrical, acoustical, mechanical, etc. techniques as may be appropriate for the type of sample and any associated conveyance means (e.g., charged particles).

Magnetic Sorting Stations

Many different magnetic field generating mechanisms may be employed to generate a magnetic field over the sorting station of the microfluidic device. In a simple case, a single permanent magnet is employed. It is positioned with respect to the flow path to provide an appropriate flux density and field gradient. Permanent magnets are made from ferromagnetic materials such as nickel, cobalt, iron, alloys of these and alloys of non-ferromagnetic materials that become ferromagnetic when combined as alloys, know as Heusler alloys (e.g., certain alloys of copper, tin, and manganese). In one specific embodiment, the permanent magnet is a cylindrical neodymium-iron-boron magnet. In another example, the magnet is an electromagnet such as a current carrying coil or a coil surrounding a paramagnetic or ferromagnetic core. In some embodiments, a controller is employed to adjust the magnetic field characteristics (the flux density, field gradient, or distribution over space) by modulating the current flowing through the coil and/or the orientation of the magnet with respect to the flowing fluid.

In some designs, a combination of magnets or magnetic field gradient generating elements is employed to generate a field of appropriate magnitude and direction. For example, one or more permanent magnets may be employed to provide an external magnetic field and current carrying conductive lines may be employed to induce a local field gradient that is superimposed on the external field. In other embodiments, “passive” elements may be employed to shape the field and produce a controlled gradient. Generally, any type of field influencing elements should be located proximate the flow path to tailor the field gradient as appropriate.

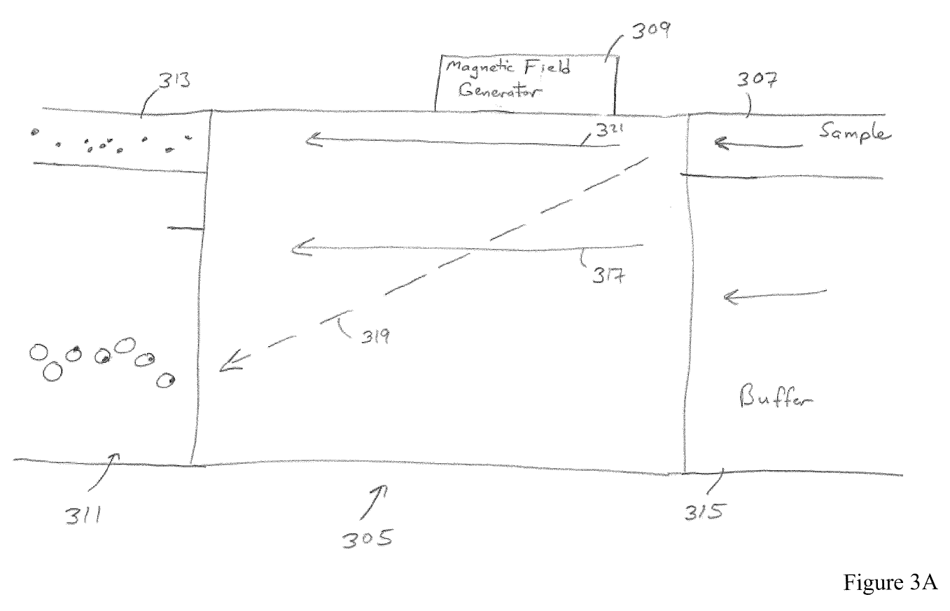

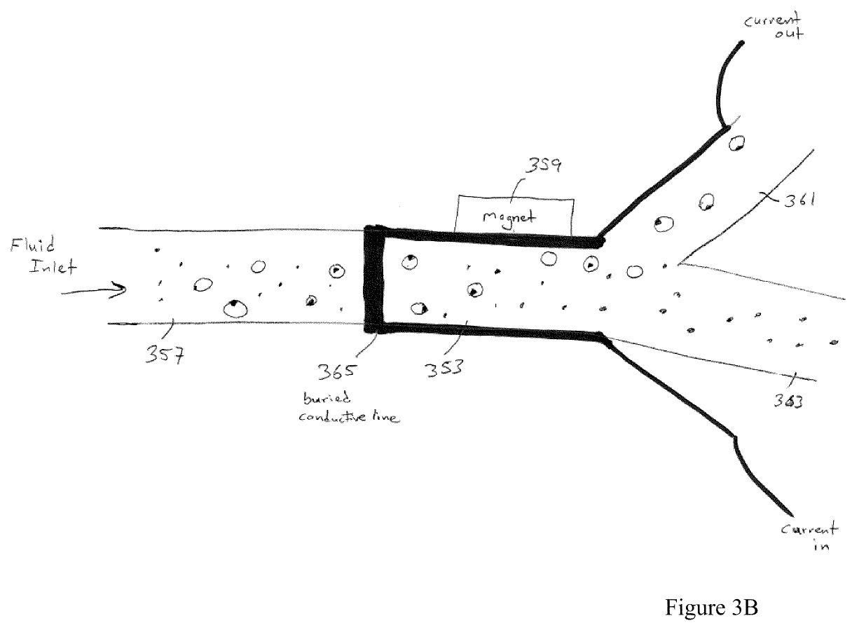

Examples of separation structures within a microfluidic device of this invention are depicted in FIGS. 3A and 3B. FIG. 3 shows a separation chamber 305 and an associated sample inlet channel 307, a magnetic field generating element 309, a magnetic particles outlet channel 311, and a non-magnetic components outlet channel 313. Each of these features is typically provided in a single microfluidic device. In operation, the sample and magnetic particles are provided to chamber 305 in a fluid medium via inlet 307. At this stage, the magnetic and non-magnetic components are commingled. A separate buffer solution may be provided to chamber 305 via a parallel inlet 315. Together the buffer and sample flow through chamber 305 in the direction shown by the arrow 317. Magnetic field gradient generating element 309 exerts a lateral force on magnetic particles while in chamber 305 causing them to deflect in the direction of arrow 319. Non-magnetic components of the sample continue to flow undeflected with the fluid to outlet 313 as indicated by arrow 321.

FIG. 3B shows an alternative magnetic separation device. This design includes both a magnet for introducing an external magnetic field and a current carrying path for producing a local field gradient. In the depicted embodiment, a fluid containing the sample and magnetic particles flows through a microchannel 357 where it encounters a portion of the channel that serves as a separation region 353. Within region 353 an external field is provided by a magnet 359 (permanent or electromagnet) and a local field is produced by current flowing through a buried metal line 365 embedded in the substrate of the device, below the flow channel 357/365. The local field introduces a magnetic field gradient that, together with the external field, applies a force on the magnetic particles flowing in region 353. At the downstream side of separation region 353 is a branch in the flow channel having one outlet 361 for receiving the magnetic particles (with sample members attached in some cases) and another outlet 363 for receiving non-magnetic components of the fluid stream. Thus, magnetic particles flowing in through separator region 353 are diverted toward the outlet 361, while other components are hydrodynamically directed toward outlet 363.

Many other microfluidic structures may be employed to effect separation of magnetic and non-magnetic components in a fluid. Some of these may employ three-dimensional flow paths, buried channels, other combinations of magnetic field generating elements, recirculation loops, etc.

Certain examples of passive magnetic field gradient generators (MFGs) will now be described. As explained, these generally include one or more MFG elements that interact with an external magnetic field to shape the field in a controlled manner, e.g., to produce a local magnetic field gradient of appropriate magnitude and direction. Pertinent parameters of MFG construction include the MFG material(s), the size and geometry of the MFG, and the orientation of the MFG with respect to the fluid flow and external magnetic field.

The material from which an MFG element is made should have a permeability that is significantly different from that of the fluid medium in the device (e.g., the buffer). In certain cases, the MFG element will be made from a ferromagnetic material. Thus, the MFG element may include at least one of iron, cobalt, nickel samarium, dysprosium, gadolinium, or an alloy of other elements that together form a ferromagnetic material. The material may be a pure element (e.g., nickel or cobalt) or it may be a ferromagnetic alloy such as an alloy of copper, manganese and/or tin.

In certain embodiments, the MFG is an array of thin metal stripes (e.g., nickel stripes) micro-patterned on a glass substrate, which becomes magnetized under the influence of an external permanent magnet. Because the stripes possess a higher permeability than the surrounding material (i.e., the buffer), a strong gradient is created at the interface. Although the magnetic flux density from the MFGs may not be strong compared to the surface of the external magnet, the gradient of the magnetic field is very large within a short distance (e.g., a few microns in some embodiments) of the line edges. As a result, the MFGs allow precise shaping of the field distribution in a reproducible manner inside microfluidic channels. The MFG element may include one or more individual magnetizable elements. The MFG may include a plurality of magnetizable elements, e.g., 2 or more, 4 or more, 5 or more, 10 or more, 15 or more, 25 or more, etc.

In designs where the magnitude of the gradient decreases rapidly with distance from the MFG, the MFG may be formed within or very close to the flow channel where sorting takes place. Therefore, in some microfluidic examples, an MFG should be located within a few micrometers of the sorting region where magnetic particles are to be deflected (e.g., within about 100 micrometers or in certain embodiments within about 50 micrometers or within about 5 micrometers of the sorting region, such as within about 2 micrometers of the sorting region). However, when large external fields are employed, the MFG design need not be so limited. Generally speaking, the MFG may be located as far away from the sorting region as about 10 millimeters. This may be the case when, for example, the external magnetic field is in the domain of about 1 Tesla or higher. Note that the large gradients afforded by such MFGs allow one to design very high throughput sorting stations with relatively large channels and consequently the capability to support large volumetric flow rates.

In certain embodiments, the MFG elements are provided within the sorting region channel; i.e., the fluid contacts the MFG structure. In certain embodiments, some or all of the MFG structure is embedded in channel walls (such as anywhere around the perimeter of the channel (e.g., top, bottom, left, or right for a rectangular channel)). Some embodiments permit MFG elements to be formed on top of or beneath the microfluidic cover or substrate.

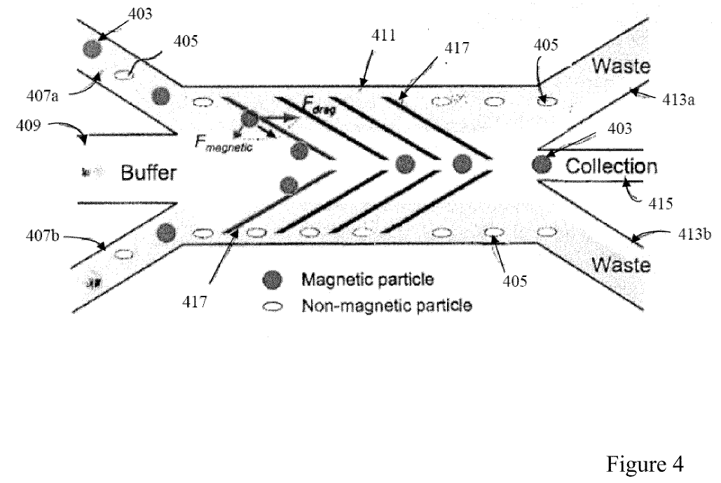

The pattern of material on or in the microfluidic substrate may take many different forms. In one embodiment it may take the form of a single strip or a collection of parallel strips. The example depicted in FIG. 4A shows four parallel strips comprising an MFG. Note that there are two MFGs in FIG. 4A, one for the magnetic particles entering the sorting region from sample channel 407a and the other for magnetic particles entering the region from sample channel 407b.

Examples of suitable dimensions for line-type MFG structures will now be presented. In certain embodiments employing ferromagnetic strips for use in sorting particles in a conventional buffer medium, the strips may be formed to a thickness of between about 1000 Angstroms and 100 micrometers. The widths of such strips may be between about 1 micrometer and 1 millimeter; e.g., between about 5 and 500 micrometers. The length, which depends on the channel dimensions and the angle of the strips with respect flow direction, may be between about 1 micrometer and 5 centimeters; e.g., between about 5 micrometers and 1 centimeter. The spacing between individual strips in such design may be between about 1 micrometer and 5 centimeters. The number of separate strips in the MFG may be between about 1 and 100. The angle of the strips with respect to the direction of flow may be between about −90° and +90°. For fractionation applications, it has been found that angles of between about 2° and 85° work well. Obviously, one or more dimensions of the MFG pattern may deviate from these ranges as appropriate for particular applications and overall design features.

In certain embodiments, the pattern of ferromagnetic material may take the form of one or more pins or pegs in the flow channel or on the substrate beside the flow channel or embedded in the substrate adjacent the flow channel.

Substrates used in microfluidic systems are the supports in which the necessary elements for fluid transport are provided. The basic structure may be monolithic, laminated, or otherwise sectioned. Commonly, substrates include one or more microchannels serving as conduits for molecular libraries and reagents (if necessary). They may also include input ports, output ports, and/or features to assist in flow control.

The substrate choice is highly dependent on the application and design of the device. Substrate materials are generally chosen for their compatibility with a variety of operating conditions. Limitations in microfabrication processes for a given material are also relevant considerations in choosing a suitable substrate. Useful substrate materials include, e.g., glass, polymers, silicon, metal, and ceramics.

Polymers are standard materials for microfluidic devices because they are amenable to both cost effective and high volume production. Polymers can be classified into three categories according to their molding behavior: thermoplastic polymers, elastomeric polymers and duroplastic polymers. Thermoplastic polymers can be molded into shapes above the glass transition temperature, and will retain these shapes after cooling below the glass transition temperature. Elastomeric polymers can be stretched upon application of an external force, but will go back to original state once the external force is removed. Elastomers do not melt before reaching their decomposition temperatures. Duroplastic polymers have to be cast into their final shape because they soften a little before the temperature reaches their decomposition temperature.

Among the polymers that may be used in microfabricated devices of this invention are polyamide (PA), polybutylenterephthalate (PBT), polycarbonate (PC), polyethylene (PE), polymethylmethacrylate (PMMA), polyoxymethylene (POM), polypropylene (PP), polyphenylenether (PPE), polystyrene (PS) and polysulphone (PSU). The chemical and physical properties of polymers can limit their uses in microfluidics devices. Specifically in comparison to glass, the lower resistance against chemicals, the aging, the mechanical stability, and the UV stability can limit the use of polymers for certain applications.

Glass, which may also be used as the substrate material, has specific advantages under certain operating conditions. Since glass is chemically inert to most liquids and gases, it is particularly appropriate for applications employing certain solvents that have a tendency to dissolve plastics. Additionally, its transparent properties make glass particularly useful for optical or UV detection.

One example of a microfluidic device suitable for sorting sample species is depicted in FIG. 4. As shown in the figure, a pattern of microfluidic channels is employed to separate magnetic particles 403 from non-magnetic particles 405. The microfluidic channels include sample inlet channels 407a and 407b, a buffer inlet channel 409, a sorting region 411, waste outlet channels 413a and 413b, and a collection channel 415. Within sorting region 411 multiple magnetic field gradient generator elements 417 are provided. In one embodiment, these are nickel strips provided within a flow channel of the sorting region itself. Not shown are one or more magnets that provide an external magnetic field in the sorting region. In one embodiment, a pair of permanent magnets is placed on the top and bottom of the sorting region. In other embodiments, one or more electromagnets may be employed to allow precise control of the field shape and homogeneity. The MFG strips interact with the field produced by the external magnet(s) to precisely shape and direct the magnetic field gradient within sorting region 411.

During operation, a buffer solution is introduced through buffer inlet channel 409 and a sample solution is introduced through sample inlet channels 407a and 407b. The sample solution may include magnetic particles and non-magnetic components from a sample being analyzed (e.g., whole cells, cell components, macromolecules, non-biological particles, etc.). Typically, the buffer contains no sample species. However, in some embodiments, the buffer may include reagents for facilitating other operations (non-sorting operations) performed in an integrated microfluidics system (e.g., sample amplification or detection). The buffer and sample solution flow through the sorting region in the laminar regime. Effectively, they flow through the sorting region as uniaxial streams, with little or no mixing. The little mixing that does occur is primarily diffusion driven.

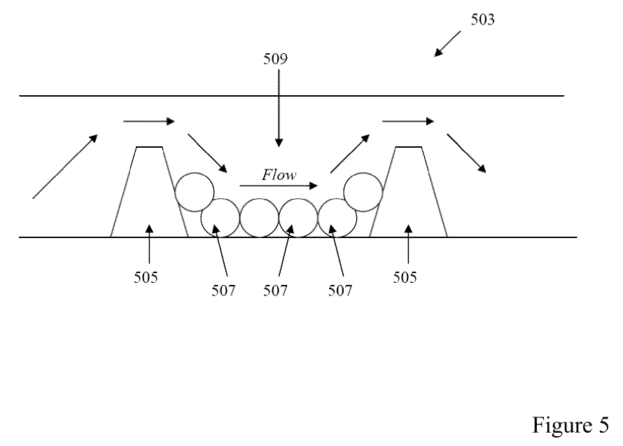

The magnetic and non-magnetic particles entering sorting region 411 through sample inlet channels 407a and 407b experience a strong magnetic field gradient imposed by the magnet and MFG strips 417. The gradient has no effect on non-magnetic materials, so the force on non-magnetic components 405 is primarily in the direction of the F_{drag} arrow in FIG. 5. This is due to the uniaxial flow of the sample solution along the outer edges of sorting region 411. Magnetic particles 403, however, experience an effective force that is a vector sum of F_{drag} and F_{magnetic}, which is the force exerted on them by the magnetic field gradient as they pass over MFG elements in the sorting region. As can be seen in the figure, the resulting force vector “guides” magnetic particles 403 along the magnetic strips and across a laminar stream boundary into the buffer stream (i.e., toward the center of sorting region 411). This process is sometimes referred to as “buffer switching.” As a consequence of buffer switching, magnetic particles 403 are directed toward collection channel 415 in a buffer stream, while non-magnetic components 405 are directed toward waste outlet channels 413a and 413b. The output of collection channel 415 contains a significantly enriched composition of the target species, as carried by the magnetic particles. As indicated, the magnetic particles are typically coated with a capture moiety.

Examples of Traps in Fluidics Systems

A variety of trapping techniques may be used to confine sorted microparticles within a space during post processing. The above discussion has emphasized trapping using magnetic forces. While this is one suitable approach, others may be employed as well in integrated fluidics devices of this invention. As explained, traps may use a mechanical, gravitational, optical, acoustic, magnetic, or electrical particle capture force. Traps also may work by physically confining microparticles to a space with walls or with boundaries defined by these forces. Any of the following types of traps may be used as described above in an integrated fluidics system. Note however that some of these traps such as the optical traps require that the particles being trapped have particular optical or electrical properties. Magnetic particles, which have been (or will be) sorted by a magnetic sorter as described above, may not possess such properties. However, their captured species may possess the necessary properties and can be trapped accordingly. Also, it is possible to introduce particles having the appropriate physical properties at an intermediate stage of the fluidic chip in order to accomplish the desired trapping.

An example of confinement in a space with walls is a weir type trap. In a weir type trap 503, one or more weirs 505 confines microparticles 507 in a space 509 bounded by the weirs, as illustrated in FIG. 5. A change in flow path cross-sectional area is used to provide a microparticle capture zone. The main flow path contains two barriers (weirs) proceeding across the bottom of the main flow path on either side of the inlet flow path. The barriers are not of sufficient height to reach the top of the main flow channel. The barriers or weirs form a trap or reservoir such that microparticles introduced into the inlet flow path tend to be captured in the cell reservoir. Any buffers or candidate compounds introduced into the main flow path upstream of the weirs or barriers will flow over the barriers and interact with the captured cells. It is contemplated that instead of using weirs or barriers to alter the flow path, the section of the main flow path immediately adjacent to the inlet flow path may be deeper or the bottom of the main flow path may be lower than the bottom of the inlet flow path or the main flow path on either side, effectively creating the reservoir. The cell may retain the particles by gravity alone or in combination with a particle capture force, which may be applied at the tops of the barriers or weirs or at the edge of the reservoir.

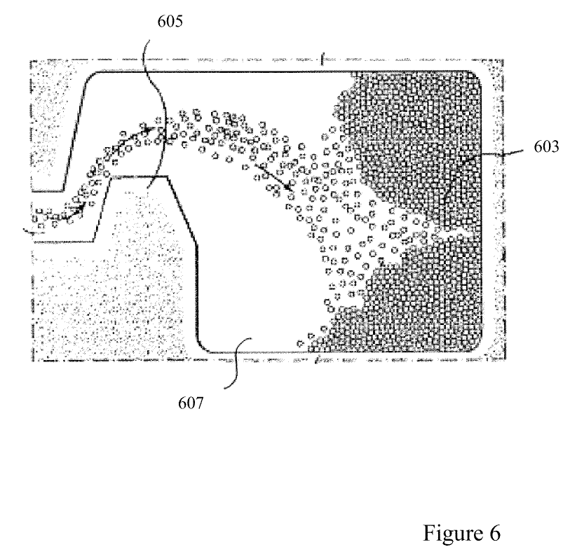

In another configuration, the microparticles 603 may cross one weir 605 into a well 607 with a controlled outlet, for example, a valve. In this configuration, microparticles are trapped by the flow as well as the weir. An example is illustrated in FIG. 6. In this example, after the post processing, a valve downstream of the inlet into the well may open to allow the microparticles to leave the trap. In addition to the weir, the microparticles may be confined by an additional particle capture force (e.g., a magnetic or dielectrophoretic force).

Another example of a particle capture force is the force associated with optical radiation. Optical radiation forces may be used to focus, trap, levitate and manipulate microparticles in a fluid medium. The optical radiation forces are gradient-forces experienced by a material (e.g., a microparticle) with a refractive index different from that of the surrounding medium when the material is placed in a light gradient. As light passes through polarizable material, it induces fluctuating dipoles. These dipoles interact with the electromagnetic field gradient, resulting in a force directed towards the brighter region of the light if the material has a refractive index larger than that of the surrounding medium. Conversely, an object with a refractive index lower than the surrounding medium experiences a force drawing it towards the darker region.

In a trap, the light gradient may be used to confine microparticles to a bottom of a well by applying a force directed toward the bottom of the well. The light gradient concept may also be used in a trap without walls, by a force directed toward a center of a space. For example, if an object has a refractive index lower than the surrounding medium and is placed in a light gradient where the center of the space is the darkest, the object would be drawn toward the center of the space. Any movement away from the center would be going against the force due to the object's refractive index.

The light field distribution and/or light intensity distribution may be produced with built-in optical elements and arrays on a microfluidic device and external optical signal sources, or may be produced with built-in electro-optical elements and arrays on a device with external structures as electrical signal sources. In the former case, when the light produced by the optical signal sources passes through the built-in optical elements and arrays, light is processed by these elements/arrays through, e.g., reflection, focusing, interference, etc. Optical field distributions are generated in the regions around the microfluidic device. In the latter case, when the electrical signals from the external electrical signal sources are applied to the built-in electro-optical elements and arrays, light is produced from these elements and arrays and optical fields are generated in the regions around the device.

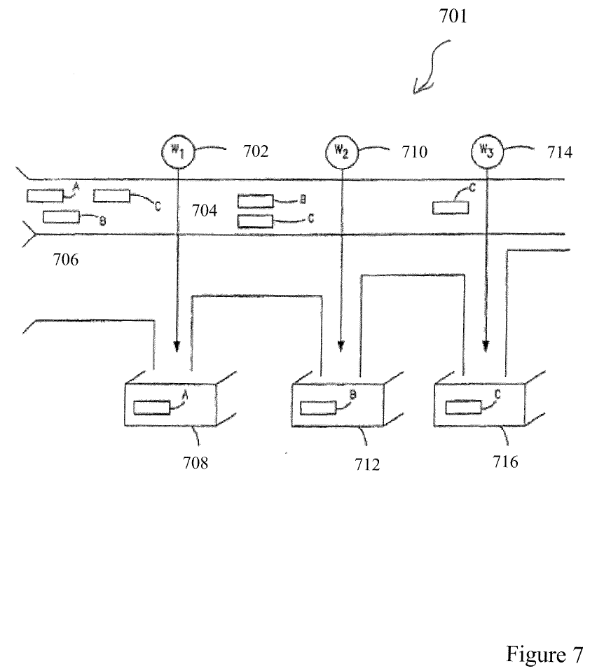

In another embodiment, a microfluidic trap using laser light may exploit the dipole created by particles having different resonance frequencies. FIG. 7 illustrates a microfluidic system 701 suitable for separately trapping microparticles A, B, and C having different resonance frequencies. This embodiment may be appropriate when the resonant frequency of particle A is higher than the resonant frequency of particle B and the resonant frequency of particle B is higher than the resonant frequency of particle C.

A first laser beam 702 may have a frequency w1 that is lower than the resonant frequency of particle A but higher than the resonant frequency of particle B. The laser beam 702 may trap particle A and move particle A from a microfluidic layer 704 to a microfluidic layer 706 to a particle trap 708. Particle B and C may remain in the microfluidic layer 704 while passing over trap 708. Another laser beam 710, directing a laser bean at a location downstream of trap 708 has a frequency w2 that is lower than the resonant frequency of particle B but higher than the resonant frequency of particle C. The laser beam 710 may trap particle B and move B from the microfluidic layer 704 to the microfluidic layer 706 to a particle trap 712. Particle C may remain in the microfluidic layer 704. Another laser beam 714 may have a frequency w3 that is lower than the resonant frequency of particle C. The laser beam 714 may trap particle C and move particle C from the microfluidic layer 704 to the microfluidic layer 706 to particle trap 716.

In one embodiment, a tunable laser provides one or more of the laser beams. The tunable laser can scan across frequencies or switch among frequencies. In an alternative embodiment, multiple lasers may be used to provide the laser beams. The laser beams may have the same frequency to provide high efficiency trapping of a target class of particles (i.e., the laser beams may be directed toward the target class simultaneously so that substantially all of the target class may be trapped in one pass). Further still, the multiple laser beams having the same frequency may be directed toward the target class sequentially so that substantially all of the target class may be trapped in a serial manner. The laser beam sweeping speed across the microfluidic flows may be fast enough so that all of the target may be trapped in one pass.

Electrical forces may also be used to trap particles. A voltage may be applied to electrodes in a microfluidic device to trap or confine ionic particles to an area around the electrode by exerting an electrostatic force. The magnitude and direction of the force depends on the charge magnitude and polarity on the particles and depends on the field magnitude and direction. The particles with positive and negative charges will be directed to electrodes with negative and positive potentials, respectively. By designing a microelectrode array in a microfluidic device, electric field distribution may be appropriately structured and realized. With DC electric fields, microparticles may be enriched, focused and moved (transported) in a microfluidic device. A suitable dielectric coating may be applied on to DC electrodes to prevent and reduce undesired surface electrochemistry and to protect electrode surfaces.

Electrokinetic force can also be used to move particles into a trap bounded by weirs or a well. Alternatively they can be used to simply hold particles in place proximate to an electrode. Shaped electrostatic fields create hydrostatic pressure (or motion) in dielectric media. When such media are fluids, a flow is produced. The mobility depends on both the particle properties (e.g., surface charge density and size) and solution properties (e.g., ionic strength, electric permittivity, and pH).

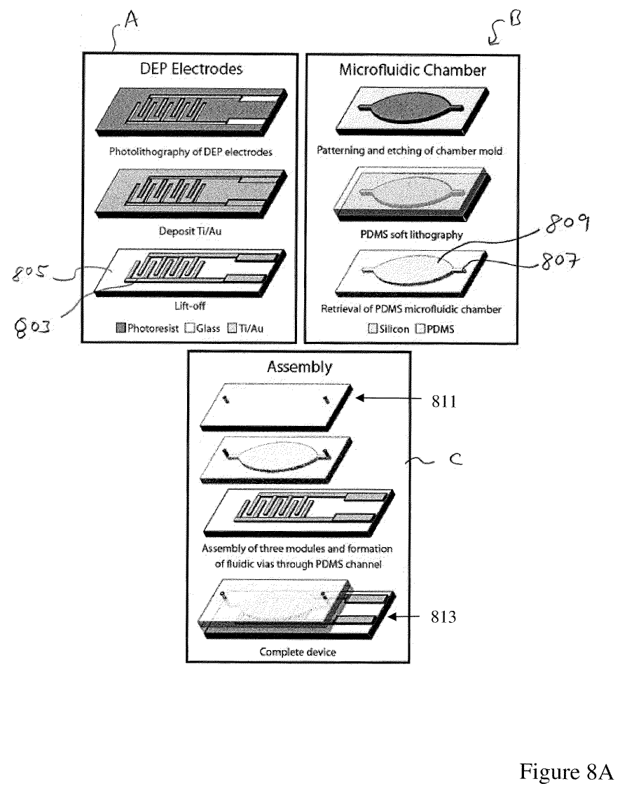

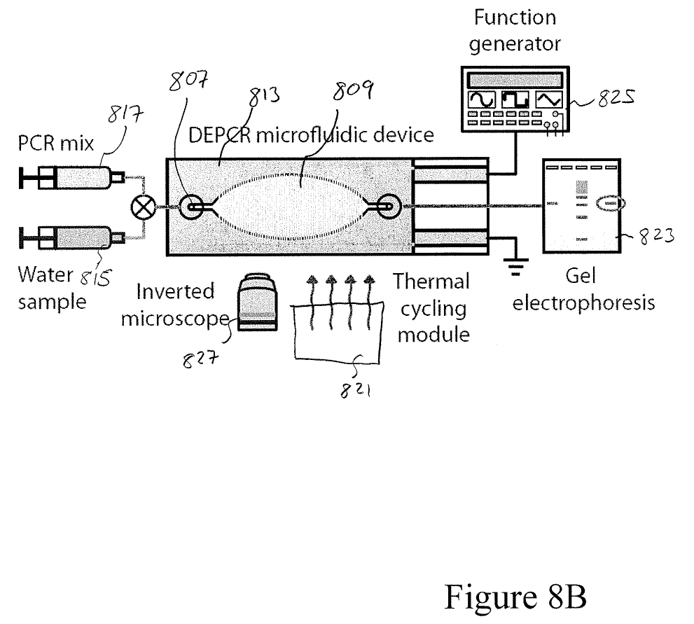

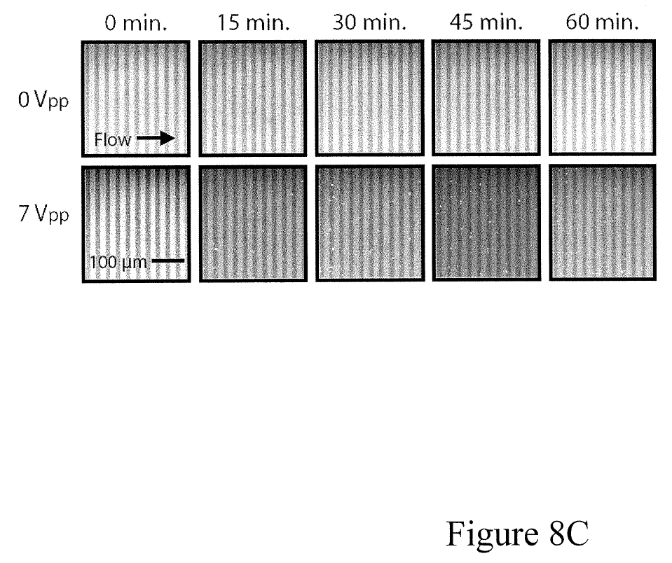

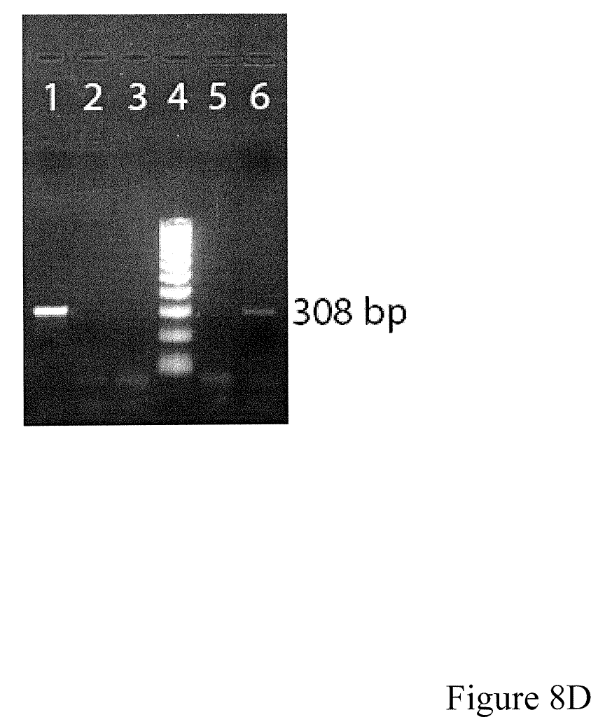

One type of electrokinetic force is dielectrophoresis (or DEP), a phenomenon in which a mechanical force is exerted on a dielectric particle when it is subjected to a non-uniform electric field. The force results from differences in the complex permittivity between a particle and its surrounding medium in a spatially varying electric field. When such a particle is placed in an electrical field, the particle will become polarized. If the applied field is non-uniform, then the interaction between the non-uniform field and the induced polarization charges will produce net force acting on the particle causing the particle to move towards a region of strong or weak field intensity. The net force acting on the particle is called dielectrophoretic force and the particle motion is dielectrophoresis. The magnitude of a dielectrophoretic force depends on the dielectric properties of the particles, particle surrounding medium, the frequency of the applied electrical field and the field distribution. In certain embodiments, a dielectrophoretic trapping module employs multiple interleaved electrodes as shown in FIG. 8A discussed below. An oscillating voltage is applied to the electrodes. An example of a fluidics device employing a dielectrophoretic trapping module is presented below with reference to FIGS. 8A to 8D.

Traveling-wave dielectrophoresis is similar to dielectrophoresis but is characterized by a traveling-electric field which interacts with the field-induced polarization and generates electrical forces acting on the particles. Particles are caused to move either with or against the direction of the traveling field. Traveling-wave dielectrophoretic forces depend on the dielectric properties of the particles and their suspending medium, the frequency and the magnitude of the traveling-field. Particles may be focused, enriched and trapped in specific regions of the electrode reaction chamber. Particles may be separated into different subpopulations over a microscopic scale. Particles may be transported over defined distances. The electrical field distribution necessary for specific particle manipulation depends on the dimension and geometry of microelectrode structures and may be designed using dielectrophoresis theory and electrical field simulation methods.

As indicated above, magnetic force is yet another particle capture force. In general, magnetic or paramagnetic particles are used with magnetic traps. When a magnetic particle made of super-dipole paramagnetic material is subjected to a magnetic field, a magnetic dipole is induced in the particle. As should be apparent from the discussion above, the particle to be trapped and processed need not be itself magnetic, it may be bound to a magnetic particle for this purpose.

The magnetic force acting on the particle is determined by the magnetic dipole moment and the magnetic field gradient. Whether there is magnetic force acting on a particle depends on the difference in the volume susceptibility between the particle and its surrounding medium. Typically, particles are suspended in a liquid, non-magnetic medium (the volume susceptibility is close to zero) thus it is necessary to utilize magnetic particles (its volume susceptibility is much larger than zero). The particle velocity under the balance between magnetic force and viscous drag depends on the particle size and viscosity of the surrounding medium. Thus to achieve sufficiently large magnetic manipulation force, the following factors should be considered: (1) the volume susceptibility of the magnetic particles should be maximized; (2) magnetic field strength should be maximized; and (3) magnetic field strength gradient should be maximized.

The magnetic particles employed in embodiments of this invention may be magnetic beads or other small objects made from a magnetic material such as a ferromagnetic material, a paramagnetic material, or a superparamagnetic material. The magnetic particles should be chosen to have a size, mass, and susceptibility that allow them to be easily diverted from the direction of fluid flow when exposed to a magnetic field in microfluidic device (balancing hydrodynamic and magnetic effects). In certain embodiments, the particles do not retain magnetism when the field is removed. In a typical example, the magnetic particles comprise iron oxide (Fe₂O₃ and/or Fe₃O₄) with diameters ranging from about 10 nanometers to about 100 micrometers. In many examples, these particles are between below one micron (e.g., 50 nm 0.5 micron) and tens of microns. However, embodiments are contemplated in which even larger magnetic particles are used. For example, it may be possible to use magnetic particles that are large enough to serve as a support medium for culturing cells.

Often, the magnetic particles will be coated with a material rendering them compatible with the microfluidics environment and allowing binding to particular species in a sample. Examples of coatings include polymer shells, glasses, ceramics, gels, etc. In certain embodiments, the coatings are themselves coated with a material that facilitates binding or physical association with sample species. For example, a polymer coating on a micromagnetic particle may be coated with an antibody, nucleic acid, avidin, or biotin.

One class of magnetic particles is the nanoparticles such as those available from Miltenyi Biotec Corporation of Bergisch Gladbach, Germany. These are relatively small particles made from coated single-domain iron oxide particles, typically in the range of about 10 to 100 nanometers diameter. They are coupled to specific antibodies, nucleic acids, proteins, etc.

Another class of magnetic particles is made from magnetic nanoparticles embedded in a polymer matrix such as polystyrene. These are typically smooth and generally spherical having diameters of about 1 to 5 micrometers. Suitable beads are available from Invitrogen Corporation, Carlsbad, Calif. These beads are also coupled to specific antibodies, nucleic acids, proteins, etc.

The manipulation of magnetic particles requires the magnetic field distribution generated over microscopic scales. One approach for generating such magnetic fields is the use of microelectromagnetic units. Such units can induce or produce magnetic field when an electrical current is applied. The switching on/off status and the magnitudes of the electrical current applied to these units will determine the magnetic field distribution. The structure and dimension of the microelectromagnetic units may be designed according to the requirement of the magnetic field distribution. Alternatively an external magnet (permanent or electromagnet) may be used.

Just like electrical force, magnetic force may be used to trap particles by moving them into a well or an area defined by walls or weirs. Conversely, magnetic force may keep particles away from a portion of a reactor chamber, such as an open valve. A magnetic field distribution may be designed such that particles would remain in the center of an area.

The last example of a particle capture force described herein is acoustic force. In one case, standing-wave acoustic field is generated by the superimposition of an acoustic wave generated from an acoustic wave source and its reflective wave. Particles in standing-wave acoustic fields experience the so-called acoustic radiation force that depends on the acoustic impedance of the particles and their surrounding medium. The acoustic impedance is the product of the density of the material and the velocity of acoustic-wave in the material. Particles with higher acoustic impedance than their surrounding medium are directed towards the pressure nodes of the standing wave acoustic field. Particles experience different acoustic forces in different acoustic field distributions.

One method to generate the acoustic wave source is to use piezoelectric material. These materials, upon applying electrical fields at appropriate frequencies, can generate mechanical vibrations that are transmitted into the medium surrounding the materials. One type of piezoelectric materials is piezoelectric ceramics. Microelectrodes may be deposited on such ceramics to activate the piezoelectric ceramic and thus to produce appropriate acoustic wave fields. Various geometry and dimensions of microelectrodes may be used according to the requirement of different applications. The reflective walls are needed to generate standing-wave acoustic field. Acoustic wave fields of various frequencies may be applied, e.g., fields at frequencies between kHz and hundred megahertz.

In another case, one could use non-standing wave acoustic field, e.g., traveling-wave acoustic field. Traveling-wave acoustic field may impose forces on particles. Particles not only experience forces from acoustic fields directly but also experience forces due to surrounding fluid because the fluid may be induced to move under traveling-wave acoustic field. Using acoustic fields, particles may be focused, concentrated, or trapped into a chamber or well defined by weirs and walls.

Another mechanism for producing forces on particles in an acoustic field is through the acoustic-induced fluid convection. An acoustic field produced in a liquid may induce liquid convection. Such convection is dependent on the acoustic field distribution, properties of the liquid, the volume and structure of the chamber in which the liquid is placed. Such liquid convection will impose forces on particles placed in the liquid and the forces may be used for manipulating particles into a trap. In one example, such manipulating forces may be exploited for enhancing mixing of liquid or mixing of particles into a liquid. A standing plane wave of ultrasound can be established by applying AC signals to the piezoelectric transducers.

Examples of Reactors and Lysis Modules in Fluidics Systems

The integrated fluidics devices disclosed herein can include one or more reactors and/or lysis modules.

Reactors

As indicated, various features may be employed in a microfluidic reactor employed in an integrated device of this invention. In certain embodiments, the reactor is placed on or downstream from a trapping module in an integrated fluidics chip. The exact design and configuration of a microfluidic reactor will depend on the type of reaction: thermal management system, micromixers, catalyst structures and a sensing system. In certain embodiments, a thermal management system includes heaters, temperature sensors and heat transfer (micro heat exchanges). In microreactors, all components can be integrated in resulting in a very precise control of temperatures which is crucial for instance in PCR for DNA amplification.

Micromixers may be used for mixing two solutions (e.g. a sample and a reagent) to make the reaction possible. In microscale systems, mixing often relies on diffusion due to the laminar behavior of fluid at low Reynolds numbers. In one embodiment, a hydrophobic material defining a hole separates two adjacent chambers. When aqueous solutions are used, the hydrophobicity of the interface permits both chambers to be filled with fluid plugs without mixing. A pressure gradient can then be applied to force fluid through the hole in the hydrophobic layer to induce diffusion between the two plugs. In one embodiment, the hole is actually a slit in which no material is removed from the intermediate dividing layer.

Catalyst structures may be employed to accelerate a chemical reaction (e.g., cross-linking or sequencing). In microreactors, the catalyst can be implemented in the form of, e.g., fixed beads, wires, thin films or a porous surface. Batch fabrication of microreactors can involve thin films and porous surface catalyst structures.

A sensing system may employ chemical microsensors or biosensors, for example. Designing a microreactor with glass or plastic provides optical access to the reaction chamber and thus, all optical measurement methods.

Lysis Modules

Before the contents of a biological cell may be analyzed, the cells to be analyzed are made to burst so that the components of the cell can be separated. The methods of cell disruption used to release the biological molecules in a cell and in a virus include, e.g., thermal energy, electric field, enzyme, sonication, and using a detergent. Mechanical forces may also be used to shear and burst cell walls.

The cell lysis may be performed by subjecting the cells trapped in a reaction chamber to pulses of high electric field strength, typically in the range of about 1 kV/cm to 10 kV/cm. The use of enzymatic methods to remove cell walls is well-established for preparing cells for disruption, or for preparation of protoplasts (cells without cell walls) for other uses such as introducing cloned DNA or subcellular organelle isolation. The enzymes are generally commercially available and, in most cases, were originally isolated from biological sources (e.g. snail gut for yeast or lysozyme from hen egg white). The enzymes commonly used include lysozyme, lysostaphin, zymolase, cellulase, mutanolysin, glycanases, proteases, mannase etc.

In addition to potential problems with the enzyme stability, the susceptibility of the cells to the enzyme can be dependent on the state of the cells. For example, yeast cells grown to maximum density (stationary phase) possess cell walls that are notoriously difficult to remove whereas midlog growth phase cells are much more susceptible to enzymatic removal of the cell wall. If an enzyme is used, it may have to be sorted and removed from the desired material before further analysis.

Sonication uses a high-frequency wave that mechanically bursts the cell walls. Ultrasound at typically 20-50 kHz is applied to the sample via a metal probe that oscillates with high frequency. The probe is placed into the cell-containing sample and the high-frequency oscillation causes a localized high pressure region resulting in cavitation and impaction, ultimately breaking open the cells. Cell disruption is available in smaller samples (including multiple samples under 200 μL in microplate wells) and with an increased ability to control ultrasonication parameters.

Detergent-based cell lysis is an alternative to physical disruption of cell membranes, although it is sometimes used in conjunction with homogenization and mechanical grinding. Detergents disrupt the lipid barrier surrounding cells by disrupting lipid:lipid, lipid:protein and protein:protein interactions. The ideal detergent for cell lysis depends on cell type and source and on the downstream applications following cell lysis. Animal cells, bacteria and yeast all have differing requirements for optimal lysis due to the presence or absence of a cell wall. Because of the dense and complex nature of animal tissues, they require both detergent and mechanical lysis to effectively lyse cells.

In general, nonionic and zwitterionic detergents are milder, resulting in less protein denaturation upon cell lysis, than ionic detergents and are used to disrupt cells when it is critical to maintain protein function or interactions. CHAPS, a zwitterionic detergent, and the Triton X™ series of nonionic detergents are commonly used for these purposes. In contrast, ionic detergents are strong solubilizing agents and tend to denature proteins, thereby destroying protein activity and function. SDS, and ionic detergent that binds to and denatures proteins, is used extensively for studies assessing protein levels by gel electrophoresis and western blotting.

A mechanical method for cell disruption uses glass or ceramic beads and a high level of agitation to shear and burst cell walls. This process works for easily disrupted cells, is inexpensive, but has integration issues for the microfluidic device. In one embodiment, beads are used in a closed chamber holding the sample and are agitated with an electric motor. In other embodiments, high pressure is applied to fluid containing the cell samples while forcing the fluid to flow through a very narrow channel. Shear between the cell and channel walls under such conditions would disrupt the cell.

Examples of Detectors in Integrated Flow Systems

In various applications envisaged for integrated Microsystems it will be desirable to detect and/or quantify the material present in a channel at one or more positions. For example, in some embodiments it will be desirable to detect the presence or absence of a target species in a fluid sample. Techniques typically utilized for quantification include, but are not limited to, optical absorbance, refractive index changes, fluorescence emission, chemiluminescence, various forms of Raman spectroscopy, electrical conductometric measurements, impedance measurements (e.g., impedance cytometry) electrochemical amperiometric measurements, acoustic wave propagation measurements.

Optical absorbance measurements are commonly employed with conventional laboratory analysis systems because of the generality of the phenomenon in the UV portion of the electromagnetic spectrum. Optical absorbance is commonly determined by measuring the attenuation of impinging optical power as it passes through a known length of material to be quantified. Alternative approaches are possible with laser technology including photo acoustic and photo thermal techniques. Such measurements can be utilized with the integrated fluidics devices discussed here, e.g., integrating optical wave guides on microfabricated devices. The use of solid-state optical sources such as LEDs and diode lasers with and without frequency conversion elements would be attractive for reduction of system size.

Refractive index detectors may also be used for quantification of flowing stream chemical analysis systems because of generality of the phenomenon but have typically been less sensitive than optical absorption. Laser based implementations of refractive index detection could provide adequate sensitivity in some situations and have advantages of simplicity. Fluorescence emission (or fluorescence detection) is an extremely sensitive detection technique and is commonly employed for the analysis of biological materials. This approach to detection has much relevance to miniature chemical analysis and synthesis devices because of the sensitivity of the technique and the small volumes that can be manipulated and analyzed (volumes in the picoliter range are feasible). For example, a 100 μL sample volume with 1 nM concentration of analyte would have only 60,000 analyte molecules to be processed and detected. There are several demonstrations in the literature of detecting a single molecule in solution by fluorescence detection. A laser source is often used as the excitation source for ultrasensitive measurements but conventional light sources such as rare gas discharge lamps and light emitting diodes (LEDs) are also used. The fluorescence emission can be detected by a photomultiplier tube, photodiode or other light sensor. An array detector such as a charge coupled device (CCD) detector can be used to image an analyte spatial distribution.

Raman spectroscopy can be used as a detection method for microfluidic devices with the advantage of gaining molecular vibrational information, but with the disadvantage of relatively poor sensitivity. Sensitivity has been increased through surface enhanced Raman spectroscopy (SERS) effects but only at the research level. Electrical or electrochemical detection approaches are also of particular interest for implementation on microfluidic devices due to the ease of integration onto a microfabricated structure and the potentially high sensitivity that can be attained. The most general approach to electrical quantification is a conductometric measurement, i.e., a measurement of the conductivity of an ionic sample. The presence of an ionized analyte can correspondingly increase the conductivity of a fluid and thus allow quantification. Amperiometric measurements imply the measurement of the current through an electrode at a given electrical potential due to the reduction or oxidation of a molecule at the electrode. Some selectivity can be obtained by controlling the potential of the electrode but it is minimal. Amperiometric detection is a less general technique than conductivity because not all molecules can be reduced or oxidized within the limited potentials that can be used with common solvents. Sensitivities in the 1 nM range have been demonstrated in small volumes (10 mL). The other advantage of this technique is that the number of electrons measured (through the current) is equal to the number of molecules present. The electrodes required for either of these detection methods can be included on a microfabricated device through a photolithographic patterning and metal deposition process. Electrodes could also be used to initiate a chemiluminescence detection process, i.e., an excited state molecule is generated via an odixation-reduction process which then transfers its energy to an analyte molecule, subsequently emitting a photon that is detected.

Acoustic measurements can also be used for quantification of materials but have not been widely used to date. One method that has been used primarily for gas phase detection is the attenuation or phase shift of a surface acoustic wave (SAW). Adsorption of material to the surface of a substrate where a SAW is propagating affects the propagation characteristics and allows a concentration determination. Selective sorbents on the surface of the SAW device are often used. Similar techniques may be useful in the devices described herein.

The mixing capabilities of the microfluidic systems lend themselves to detection processes that include the addition of one or more reagents. Derivatization reactions are commonly used in biochemical assays. For example, amino acids, peptides and proteins are commonly labeled with dansylating reagents or o-phthaldialdehyde to produce fluorescent molecules that are easily detectable. Alternatively, an enzyme can by used as a labeling molecule and reagents, including substrates, could be added to provide an enzyme amplified detection scheme, i.e., the enzyme produces a detectable product. A third example of a detection method that can benefit from integrated mixing methods is chemiluminescence detection. In these types of detection scenarios, a reagent and a catalyst are mixed with an appropriate target molecule to produce an excited state molecule that emits a detectable photon.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); and the like.

### Experimental PCR Example

A dielectrophoretic trapping and PCR (DEPCR) module was fabricated and tested. Such a module can be integrated with a magnetic species sorting station of a type as described above.

This example describes a Dielectrophoresis-Enhanced Polymerase Chain Reaction (DEPCR) device: a microfluidic system capable of performing dielectrophoretic enrichment of waterborne pathogens integrated with in situ cell lysis and gene-specific DNA amplification. The DEPCR devices was used to detect physiologically-relevant concentrations of microorganisms (˜100 cells mL⁻¹) from commercial drinking water samples.

Escherichia coli MC1061 cells with resistance to chloramphenicol (CM) were doped into a sample of commercially-available bottled drinking water (Arrowhead™, Lake Arrowhead, Calif.). The cells were transformed with a plasmid containing the gene for green fluorescent protein (GFP) under the control of the arabinose promoter to facilitate visualization. The cells were grown overnight at 37° C. on agar plates, after which colonies were randomly picked, and suspended in Luria-Bertani (LB) broth with 34 μg mL⁻¹ CM (Sigma, St. Louis, Mo.). These cell cultures were subsequently sub-cultured 1:50 into fresh LB medium for 2 hours at 37° C. Expression of GFP was induced by the addition of 0.02% L-arabinose, after which the cultures were incubated for an additional 2 hours at 37° C. A 1 mL aliquot of the cell culture was washed in deionized (DI) water, and centrifuged for 5 minutes at 5000 rpm on a tabletop centrifuge (Eppendorf, Westbury, N.Y.). The bacterial pellet was resuspended in 1 mL of drinking water, and cell density was quantified by an optical density measurement at 600 nm with a tabletop spectrophotometer (Biophotometer, Eppendorf, Westbury, N.Y.). Finally, the cell suspension was serially diluted in drinking water prior to the DEPCR experiment.

A PCR reaction mix (120 μL) contained 60 μL HotStarTaq PCR Master Mix (Qiagen, Hilden, Germany), 3.6 μL 10 μM E. coli K12/O157:H7 forward primer (5′-AAG AAA GGC AAG CAG GAA CA-3′; SEQ ID NO:1), 3.6 μL 10 μM E. coli K12/O157:H7 reverse primer (5′-TTG TCG TTC AGT TCG CTG AC-3′; SEQ ID NO:2), 1 μL 12 μg μL⁻¹ bovine serum albumin (BSA) solution (Fisher Scientific, Tustin, Calif.), 12 μL 20% polyethyleneglycol (PEG) 8000 (Sigma-Aldrich, St. Louis, Mo.), and 39.8 μL nuclease-free water. The final concentrations for the primers, BSA, and PEG 8000 were 0.3 μM, 0.1 μg μL⁻¹, and 2%, respectively. The PCR reaction mix was degassed inside a tabletop desiccator connected to a floor pump for 10 minutes prior to injection into the DEPCR device. Before introducing the PCR reaction mix, a BSA solution (1 μg μL⁻¹) and a PCR wash buffer were injected into the device to passivate device surfaces and chemically enhance the surface hydrophilicity. The PCR wash buffer contained 10% v/v 10×PCR assay buffer (pH 9.0, 100 mM Tris-HCl and 500 mM KCl; Fisher Scientific, Fair Lawn, N.J.), 4% v/v 25 mM MgCl₂ (Fisher Scientific, Fair Lawn, N.J.), and 86% v/v DI water.

The DEPCR device was fabricated using a modular architecture with three components: the DEP electrode component (glass), the microfluidic component (polydimethylsiloxane; PDMS) and the interconnect component (glass), each of which were processed independently prior to assembly. The DEP electrodes on the electrode chip were patterned with standard photolithography and lift-off processes on a 500-μm-thick, 4-inch borosilicate glass wafer (Pyrex 7740, Corning, N.Y.) using a mask aligner (MJB3, SUSS MicroTec AG, Garching, Germany). The lift-off was performed first with an electron-beam deposition of 20 nm of titanium and 180 nm of gold, followed by immersion and sonication of the wafer in an acetone bath. The wafer was diced into individual DEP electrode components using a dicing saw (Disco DAD-2H/6, Tokyo, Japan). The microfluidic component was fabricated via PDMS soft lithography. The negative-tone master mold of the microfluidic layer was fabricated on a 4-inch silicon (Si) wafer by photolithography and deep reactive ion etching (SLR-770, Plasmatherm, St. Petersburg, Fla.), which produced 60-μm-tall mesas. 500-μm-thick spacers were glued to the mold to define the thickness of the PDMS layer. Subsequently, the surface of the Si mold was cleaned with 49% hydrofluoric acid (Fisher Scientific, Fair Lawn, N.J.) for 1 minute. Uncured PDMS mixture, composed of 10 parts base resin mixed with 1 part curing agent (Sylgard 184, Dow Corning, N.Y.), was degassed and poured onto the Si master. To ensure that the top of the PDMS layer was flat, a separate glass wafer coated with hard-baked photoresist was used as the backing layer during the replication step. After curing at 70° C. for 3 hours, the patterned PDMS layer was peeled off from the Si mold and cut into individual chips. The glass interconnect wafer was first drilled with a custom CNC mill (Flashcut CNC, Menlo Park, Calif.) to establish inlet and outlet and subsequently diced into individual interconnect components. The three modular components were each cleaned prior to assembly. The electrode and interconnect components were immersed and sonicated in acetone, isopropanol, and DI water for two minutes each, followed by a cleaning step in piranha (9:1 solution of H₂SO₄ and H₂O₂). The microfluidic component was processed by a similar protocol without the piranha step. The cleaned components were manually aligned and assembled into a glass-PDMS-glass sandwich structure which formed the chip. During this assembly step, microfluidic connections were established by coring inlet and outlet holes through the PDMS microfluidic chip with a custom-made coring tool. The device was subsequently treated in a UV-ozone chamber (PR-100, UVP, Upland, Calif.) to irreversibly bond PDMS to glass. Finally, Tygon tubing (ID 0.03 inch, OD 0.09 inch; Fisher Scientific, Fair Lawn, N.J.) was glued to the inlet and outlet of the device with epoxy.

The resulting DEPCR chip was 30 mm long by 9 mm wide and incorporated 210 pairs of electrodes, each of which is 15 μm wide at a pitch of 30 μm. The electrodes were made of a 200 Å-thick titanium layer and an 1800 Å-thick layer of gold fabricated using standard microfabrication techniques as illustrated in FIG. 8A. Gold was selected due to its favorable electrolysis characteristics and inertness to PCR reagents. An elliptical PDMS fluidic chamber was 60 μm in height, defining a volume of 3.5 μL. To prevent evaporation of sample and reagents during the on-chip PCR reaction, custom plugs covered with Teflon tape were used to tightly seal the fluidic connections.

The DEPCR device was fabricated with a modular architecture with three components using a glass-PDMS-glass microfabrication process. An electrode component was fabricated as shown in panel A of FIG. 8A by patterning Ti/Au DEP electrodes 803 on a glass substrate 805. The microfluidic component was fabricated as shown in panel B by PDMS soft lithography, and it defined the microfluidic channels 807 and chamber 809. The interconnect component 811 establishes the microfluidic connections to the device. The three components were assembled into a functioning microfluidic device 813 as shown in panel C of FIG. 8A.

As shown in FIG. 8B, a drinking water sample doped with bacteria was injected into the DEPCR device 813, where positive DEP forces trap the cells at the interdigitated DEP electrodes 803 (shown in FIG. 8A) in the device chamber. Specifically, samples were continuously injected into the DEPCR device inlet at 200 μL h⁻¹ with a programmable syringe pump 815 (PHD 2000, Harvard Apparatus, Holliston, Mass.). The low Reynolds number within the device (Re_{DEPCR}<0.2) ensured laminar flow with zero turbulence. The interdigitated microelectrodes 803 within the device were powered by an external function generator 825 (AFG320, Tektronix, Richardson, Tex.), at 7.0 V peak to peak (Vpp) at 1 kHz to create a DEP force field at the edges of the electrodes 803. Amplitude and frequency were monitored by a digital oscilloscope (54622A, Agilent Technologies, Palo Alto, Calif.).

The DEP force responsible for the trapping and concentration of bacteria originates from the difference in the complex permittivity between the target bacterium (∈_{bac}*) and the suspension medium (∈ₘ*). Assuming a spherical geometry, the time-averaged DEP force on a cell of radius r_{bac} can be approximated as:

\vec{F}_{DEP}=2π∈ₘr_{bac}³Re(CM(f))∇|Eᵣₘₛ|² (1)

where ∈ₘ is the permittivity of the medium, CM is the Clausius-Mossotti factor, and f and Eᵣₘₛ are the frequency and the root-mean-square value of the applied electric field, respectively. The polarity of the force is governed by CM, where:

$$CM ( f ) = \frac{{ɛ}_{bac}^{*} ( f ) - {ɛ}_{m}^{*} ( f )}{{ɛ}_{bac}^{*} ( f ) + 2 {ɛ}_{m}^{*} ( f )} \qquad ( 2 )$$

Because both ∈_{bac}* and ∈ₘ* are functions of frequency, the direction of the force exerted on the target cells can be controlled by tuning the frequency of the applied voltage. For example, making Re (CM)>0 (i.e., positive DEP) exerts an attractive force on the cell, whereas making Re (CM)<0 (i.e., negative DEP) generates a repulsive force. In the described experiment, the frequency of the applied voltage was optimized to maximize the positive DEP force.

Cells entering the elliptical fluidic chamber were continuously trapped in the device by the dielectrophoresis force, resulting in a significant increase in local concentration. The trapped cells could be observed directly through an inverted epifluorescence microscope 827 (Nikon Eclipse TE2000S, Nikon Instruments, Inc., Melville, N.Y.). After a period of continuous trapping, PCR buffers and reaction mix are injected into the device via a syringe pump 817 through the inlet 807, and thermal cell lysis and PCR amplification were performed directly within the trapping chamber 809. The thermal cycling for the PCR reaction was performed with a custom thermal cycling apparatus 821 using a thin film heater element (ASI5900, Minco, Minneapolis, Minn.) in closed loop feedback with a commercial temperature controller (CN8201, Omega, Stamford, Conn.). This thermal module design allowed parallel operation of multiple DEPCR devices, enabling simultaneous collection of data from positive and negative control samples in a single experimental run. After the reaction, the PCR amplicons were extracted by pipette and analyzed off-chip via gel electrophoresis.

The efficiency of bacterial cell trapping was measured with a suspension of E. coli MC1061 cells expressing GFP to enhance visualization, doped into deionized water at a concentration of 1×10⁵ cells mL⁻¹. Initially, the degree of non-specific adhesion of the bacteria to the chamber surfaces was determined by injecting the sample into the device at 200 μL h⁻¹ with the electrodes turned-off. During this experiment, a small steel bead was placed in the syringe and the pump was placed on a custom-made rocking stage to ensure uniform distribution of E. coli in the sample mixture. Time-lapse micrographs of a 0.1 mm² active area after 1 hour of sample flow showed negligible non-specific adhesion (FIG. 8C, top row). When the electrodes were powered with a sine-wave AC voltage of 7 Vpp at 1 kHz, one could observe immediate trapping of the cells at the edges of the electrodes, where the electric field gradient is the highest (FIG. 8C, bottom row).

To demonstrate the integration of DEP trapping and PCR amplification in a single chamber, a sample containing ˜100 cells mL⁻¹ doped into commercially-available drinking water (electrical conductivity of 7 mS m⁻¹) was prepared. The sample was continuously injected into the device for 12 hours at a flow rate of 200 μL h⁻¹, with the DEP electrodes powered at 7.0 Vpp at 1 kHz. No electrolysis was detected under these experimental conditions, and even after many hours of trapping, the cells continued to show a high level of GFP expression (data not shown). As a negative control, a drinking water sample doped with ˜1000 cells mL⁻¹ was injected into a separate DEPCR device under the same conditions without powering the DEP electrodes. After the trapping step, 1 μg μL⁻¹ BSA solution, PCR wash buffer, and PCR reaction mix were sequentially injected into both devices at 100 μL h⁻¹; the BSA passivates the PDMS surfaces and PCR wash buffer enhances its hydrophilicity.

The integration of DEP trapping and PCR into a single chamber is especially advantageous for the detection of rare targets, as it enables in situ sample concentration and sequence-specific genetic detection with minimal loss of target species resulting from sample preparation and transport. By plating the population eluted during the loading of the PCR reagents, the total sample loss was determined to be ˜8% (data not shown). After loading the reaction mix, the cells were thermally lysed at 95° C. for 15 minutes, after which the lysates in both devices were subjected to PCR amplification for 35 cycles of 94° C. (melting) for 20 seconds, 55° C. (annealing) for 20 seconds and 72° C. (elongation) for 30 seconds, followed by a final 10 minute extension at 72° C. These reaction conditions were designed for the amplification of a segment of the E. coli gyrase B (gyrB) gene, with an expected product length of 308 bp. After the PCR reaction in the device, the amplicons were extracted and analyzed via electrophoresis on a 1% agarose gel enhanced with GelStar (Lonza, Rockland, Me.), a nucleic acid gel stain for working at lower limits of detection.

The expected 308 bp PCR product was readily detectable in the output from the DEPCR device operated with the target concentration of 100 cells mL⁻¹ (FIG. 8D, lane 6 (compare with the positive control shown in lane 1)). In addition, negative control experiments with no DNA template (lane 2) or with DNA from a different bacterial species, Salmonella enterica, confirmed the target specificity of the system (lane 3). Furthermore, even at target concentrations which were ten fold higher (1000 cells mL⁻¹), no amplification was evident in the output when the DEP electrodes were turned off during the operation (lane 5). These results clearly indicate the effectiveness of DEP concentration, and demonstrate that DEPCR device is capable of specific detection of bacteria at concentrations of 100 cells mL⁻¹.

This experimental example demonstrated an integrated device capable of continuous, label-free, electrokinetic concentration of microorganisms from commercial drinking water, with on-chip PCR amplification for the detection of specific genes from low target concentrations. The DEPCR device was able to operate continuously for more than 12 hours, and its limit of detection (LOD) was below ˜100 cells mL⁻¹, a physiologically relevant dose for many waterborne pathogens.

The disclosed DEPCR device takes advantage of a number of useful features of microfluidics technology. The microfabrication of the 210 DEP electrode pairs enables the controlled generation of large electric field gradients that create reproducible DEP trapping forces. In addition, the micro-molding of the PDMS chambers provides accurate control of the chamber height, ensuring that the electric field gradients can reach the entire volume of the chamber.

An elliptical chamber was found to reproducibly provide superior results, possibly because it allows smoother transitions in the flow pattern at the corners of the chamber. Few if any bubbles were observed. The experimental example considered possible diminished PCR efficiencies in PDMS devices due to unwanted adsorption of DNA polymerase to the device surfaces. It was found that the addition of 0.1 μg μL⁻¹ BSA and 2% PEG 8000 to the PCR mix greatly enhanced the on-chip PCR performance. Possibly the BSA and PEG 8000 reagents competitively block DNA polymerase adsorption to PDMS, freeing the polymerase and increasing the efficiency of the PCR reactions. PEG 8000 provides an additional advantage by increasing the boiling point of the PCR reaction mix, reducing the likelihood of air bubble formation.

In summary, the disclosed DEPCR system exemplifies a microsystem that integrates multiple functions onto a single chip to achieve a significant enhancement in performance. This detection architecture could easily be adapted for the automated screening of samples containing multiple pathogens and virulence genes via multiplexed PCR. In certain embodiments, the DEPCR system is integrated with biosensors to directly detect amplicons within the chip.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A fluidic sorting device comprising:
(a) a substrate comprising a plurality of fluidic modules;
(b) a sorting station comprising a magnetic field concentrator for exerting magnetic force on a sample in a fluid medium to divert magnetic particles in the sample and thereby sort magnetic and non-magnetic entities in the fluid medium;
(c) a trapping module fluidically coupled to the sorting station and located downstream therefrom and comprising (i) a valve for controlling flow of the fluid medium between the sorting station and the trapping module, (ii) an inlet for delivering a reagent, and (iii) a structure for capturing and immobilizing, at least temporarily, magnetic particles or a particular species in the sample,
wherein the trapping module is configured to perform one or more of the following functions: releasing the magnetic particles from sample species specifically bound to the magnetic particles, and washing trapped species in the sample; and
(d) an additional module fluidically coupled to the trapping module and located downstream therefrom and comprising a valve for controlling flow of the fluid medium between the trapping module and the additional module,
wherein the additional module is configured to perform one or more of the following functions: species detection and nucleic acid amplification, and
wherein the sorting station, the trapping module, and the additional module are each provided on the substrate.

2. The fluidic sorting device of claim 1, wherein the sorting station comprises at least one inlet channel for receiving the fluid medium with the magnetic particles, and at least one outlet channel for delivering the fluid medium enriched in or depleted of said magnetic particles.

3. The fluidic sorting device of claim 1, wherein the magnetic field concentrator in the sorting station comprises one or more elements made from a material having a permeability that is significantly different from that of the fluid medium in the device.

4. The fluidic sorting device of claim 3, wherein the magnetic field concentrator comprises a pattern of ferromagnetic material arranged to interact with an external magnetic field and thereby produce a strong magnetic field gradient in the sorting station.

5. The fluidic sorting device of claim 1, wherein the sorting station comprises an inlet configured to deliver buffer to the sorting station and an inlet configured to deliver sample to sorting station.

6. The fluidic sorting device of claim 1, wherein the trapping module comprises ferromagnetic structures for concentrating a magnetic field in the trapping module, and wherein the trapping module is configured to release the magnetic particles from sample species specifically bound to the magnetic particles.

7. The fluidic sorting device of claim 6, wherein the ferromagnetic structures are located preferentially toward an outlet of the trapping module.

8. The fluidic sorting device of claim 1, wherein the reagent for use in the trapping module is a buffer.

9. The fluidic sorting device of claim 1, wherein the trapping station is configured to label the trapped species from the sample.

10. The fluidic sorting device of claim 1, wherein the trapping module is configured to release specifically bound sample species from said magnetic particles.

11. The fluidic sorting device of claim 1, wherein the trapping module is configured to perform a nucleic acid amplification process.

12. The fluidic sorting device of claim 11, wherein the trapping module is configured to perform lysis of trapped species from the sample and subsequently perform PCR on nucleic acids released during the lysis.

13. The fluidic sorting device of claim 1, wherein the additional module comprises a detector.

14. The fluidic sorting device of claim 1, wherein the additional module is configured to perform a nucleic acid amplification technique.

15. The fluidic sorting device of claim 1, further comprising a controller coupled to the substrate for controlling and synchronizing the operations of the sorting station, the trapping module, and the additional module.

16. A method of processing a sample, the method comprising:
(a) providing the sample to a fluidic sorting device on a substrate, wherein the sample includes magnetic particles capable of specifically binding one or more species suspected of being contained in the sample;
(b) magnetizing a magnetic field concentrator to divert and thereby concentrate the magnetic particles in the sample;
(c) delivering the magnetic particles and bound species, if any, from the sample to a trapping module;
(d) washing magnetic particles and/or releasing bound species, if any, from the sample while in the trapping module; and
(e) delivering the bound species, if any, to an additional module where the bound species, if any, are detected and/or reacted with a reagent.

17. The method of claim 16, wherein in the additional module, nucleic acids associated with the bound species, if any, are amplified.

18. The method of claim 16, further comprising, prior to (a), labeling the one or more species, if any, with the magnetic particles.

19. The method of claim 16, further comprising performing one or more of the following operations in the trapping module: nucleic acid amplification, a restriction enzyme reaction, nucleic acid sequencing, target labeling, chromatin immunoprecipitation, crosslinking, and cell culture.

20. The method of claim 16, further comprising performing lysis of sample species, if any, in the additional module.

21. A microfluidic sorting device comprising:
(a) a magnetic field gradient generator for exerting a magnetic force on a sample to divert magnetic particles in the sample and thereby separate them from other components; and
(b) a pre-processing and/or a post-processing trapping station integrated on the microfluidic sorting device with the magnetic field gradient generator.

22. The microfluidic sorting device of claim 21, wherein the pre-processing station comprises a labeling station for labeling target species in the sample with magnetic particles capable of specifically binding the target species.

23. The microfluidic sorting device of claim 21, wherein the post-processing station comprises a detection station for detecting the target species.

24. The microfluidic sorting device of claim 21, wherein at least one of the pre-processing station or the post-processing station comprises one or more of the following: (a) an enrichment module, (b) a reaction module, (c) a detection module, and (d) a lysis module for lysing cells or disrupting viral protein coats.

25. The microfluidic sorting device of claim 21, wherein at least one of the pre-processing station or the post-processing station is designed or configured to perform genomic analysis, amplification of a nucleic acid, gene expression analysis, enzymatic activity assays, receptor binding assays, or ELISA assays.


SEQUENCE LISTING (2 sequences)

SEQ ID NO: 1 - DNA, 20 bp, Artificial Sequence
  Primer
aagaaaggcaagcaggaaca

SEQ ID NO: 2 - DNA, 20 bp, Artificial Sequence
  Primer
ttgtcgttcagttcgctgac